SEQUENCE LISTING (28 sequences)

SEQ ID NO: 1 - protein, 306 aa, Synthetic construct
MDEFEMIKRNTSEIISEEELREVLKKDEKSAGIGFEPSGKIHLGHYLQIKKMIDLQNAGFDIIIVLADLHAYLNQKGELDEIRKIGDYNKKVFEAMGLKAKYVYGSEFQLDKDYTLNVYRLALKTTLKRARRSMELIAREDENPKVAEVIYPIMQVNMDHYYGVDVTVGGMEQRKIHMLARELLPKKVVCIHNPVLTGLDGEGKMSSSKGNFIAVDDSPEEIRAKIKKAYCPAGVVEGNPIMEIAKYFLEYPLTIKGPEKFGGDLTVNSYEELESLFKNKELHPMRLKNAVAEELIKILEPIRKRL

SEQ ID NO: 2 - protein, 306 aa, Synthetic construct
MDEFEMIKRNTSEIISEEELREVLKKDEKSAGIGFEPSGKIHLGHYLQIKKMIDLQNAGFDIIIVLADLHAYLNQKGELDEIRKIGDYNKKVFEAMGLKAKYVYGSEFQLDKDYTLNVYRLALKTTLKRARRSMELIAREDENPKVAEVIYPIMQVNGSHYYGVDVYVGGMEQRKIHMLARELLPKKVVCIHNPVLTGLDGEGKMSSSKGNFIAVDDSPEEIRAKIKKAYCPAGVVEGNPIMEIAKYFLEYPLTIKGPEKFGGDLTVNSYEELESLFKNKELHPMRLKNAVAEELIKILEPIRKRL

SEQ ID NO: 3 - protein, 306 aa, Synthetic construct
MDEFEMIKRNTSEIISEEELREVLKKDEKSAGIGFEPSGKIHLGHYLQIKKMIDLQNAGFDIIIVLADLHAYLNQKGELDEIRKIGDYNKKVFEAMGLKAKYVYGSEFQLDKDYTLNVYRLALKTTLKRARRSMELIAREDENPKVAEVIYPIMQVNYDHYYGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKGPEKFGGDLTVNSYEELESLFKNKELHPMRLKNAVAEELIKILEPIRKRL

SEQ ID NO: 4 - protein, 306 aa, Synthetic construct
MDEFEMIKRNTSEIISEEELREVLKKDEKSAGIGFEPSGKIHLGHYLQIKKMIDLQNAGFDIIIVLADLHAYLNQKGELDEIRKIGDYNKKVFEAMGLKAKYVYGSEFQLDKDYTLNVYRLALKTTLKRARRSMELIAREDENPKVAEVIYPIMQVNCDHYGGVDVLVGGMEQRKIHMLARELLPKKVVCIHNPVLTGLDGEGKMSSSKGNFIAVDDSPEEIRAKIKKAYCPAGVVEGNPIMEIAKYFLEYPLTIKGPEKFGGDLTVNSYEELESLFKNKELHPMRLKNAVAEELIKILEPIRKRL

SEQ ID NO: 5 - protein, 306 aa, Synthetic construct
MDEFEMIKRNTSEIISEEELREVLKKDEKSAGIGFEPSGKIHLGHYLQIKKMIDLQNAGFDIIIVLADLHAYLNQKGELDEIRKIGDYNKKVFEAMGLKAKYVYGSEFQLDKDYTLNVYRLALKTTLKRARRSMELIAREDENPKVAEVIYPIMQVNISHYDGVDVLVGGMEQRKIHMLARELLPKKVVCIHNPVLTGLDGEGKMSSSKGNFIAVDDSPEEIRAKIKKAYCPAGVVEGNPIMEIAKYFLEYPLTIKGPEKFGGDLTVNSYEELESLFKNKELHPMRLKNAVAEELIKILEPIRKRL

SEQ ID NO: 6 - protein, 306 aa, Synthetic construct
MDEFEMIKRNTSEIISEEELREVLKKDEKSAGIGFEPSGKIHLGHYLQIKKMIDLQNAGFDIIIVLADLHAYLNQKGELDEIRKIGDYNKKVFEAMGLKAKYVYGSEFQLDKDYTLNVYRLALKTTLKRARRSMELIAREDENPKVAEVIYPIMQVNGYHYRGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKGPEKFGGDLTVNSYEELESLFKNKELHPMRLKNAVAEELIKILEPIRKRL

SEQ ID NO: 7 - protein, 306 aa, Methanocaldococcus jannaschii
MDEFEMIKRNTSEIISEEELREVLKKDEKSAYIGFEPSGKIHLGHYLQIKKMIDLQNAGFDIIILLADLHAYLNQKGELDEIRKIGDYNKKVFEAMGLKAKYVYGSEFQLDKDYTLNVYRLALKTTLKRARRSMELIAREDENPKVAEVIYPIMQVNDIHYLGVDVAVGGMEQRKIHMLARELLPKKVVCIHNPVLTGLDGEGKMSSSKGNFIAVDDSPEEIRAKIKKAYCPAGVVEGNPIMEIAKYFLEYPLTIKRPEKFGGDLTVNSYEELESLFKNKELHPMDLKNAVAEELIKILEPIRKRL

SEQ ID NO: 8 - DNA, 924 bp, Synthetic construct
  source 1..924: mol_type = other DNA
atggacgaatttgaaatgataaagagaaacacatctgaaattatcagcgaggaagagttaagagaggttttaaaaaaagatgaaaaatctgctgggatagggtttgaaccaagtggtaaaatacatttagggcattatctccaaataaaaaagatgattgatttacaaaatgctggatttgatataattatagttttggctgatttacacgcctatttaaaccagaaaggagagttggatgagattagaaaaataggagattataacaaaaaagtttttgaagcaatggggttaaaggcaaaatatgtttatggaagtgaattccagcttgataaggattatacactgaatgtctatagattggctttaaaaactaccttaaaaagagcaagaaggagtatggaacttatagcaagagaggatgaaaatccaaaggttgctgaagttatctatccaataatgcaggttaatatggatcattattatggcgttgatgttactgttggagggatggagcagagaaaaatacacatgttagcaagggagcttttaccaaaaaaggttgtttgtattcacaaccctgtcttaacgggtttggatggagaaggaaagatgagttcttcaaaagggaattttatagctgttgatgactctccagaagagattagggctaagataaagaaagcatactgcccagctggagttgttgaaggaaatccaataatggagatagctaaatacttccttgaatatcctttaaccataaaagggccagaaaaatttggtggagatttgacagttaatagctatgaggagttagagagtttatttaaaaataaggaattgcatccaatgcgcttaaaaaatgctgtagctgaagaacttataaagattttagagccaattagaaagagattataataa

SEQ ID NO: 9 - DNA, 924 bp, Synthetic construct
  source 1..924: mol_type = other DNA
atggacgaatttgaaatgataaagagaaacacatctgaaattatcagcgaggaagagttaagagaggttttaaaaaaagatgaaaaatctgctgggatagggtttgaaccaagtggtaaaatacatttagggcattatctccaaataaaaaagatgattgatttacaaaatgctggatttgatataattatagttttggctgatttacacgcctatttaaaccagaaaggagagttggatgagattagaaaaataggagattataacaaaaaagtttttgaagcaatggggttaaaggcaaaatatgtttatggaagtgaattccagcttgataaggattatacactgaatgtctatagattggctttaaaaactaccttaaaaagagcaagaaggagtatggaacttatagcaagagaggatgaaaatccaaaggttgctgaagttatctatccaataatgcaggttaatgggtctcattattatggcgttgatgtttatgttggagggatggagcagagaaaaatacacatgttagcaagggagcttttaccaaaaaaggttgtttgtattcacaaccctgtcttaacgggtttggatggagaaggaaagatgagttcttcaaaagggaattttatagctgttgatgactctccagaagagattagggctaagataaagaaagcatactgcccagctggagttgttgaaggaaatccaataatggagatagctaaatacttccttgaatatcctttaaccataaaagggccagaaaaatttggtggagatttgacagttaatagctatgaggagttagagagtttatttaaaaataaggaattgcatccaatgcgcttaaaaaatgctgtagctgaagaacttataaagattttagagccaattagaaagagattataataa

SEQ ID NO: 10 - DNA, 924 bp, Synthetic construct
  source 1..924: mol_type = other DNA
atggacgaatttgaaatgataaagagaaacacatctgaaattatcagcgaggaagagttaagagaggttttaaaaaaagatgaaaaatctgctgggatagggtttgaaccaagtggtaaaatacatttagggcattatctccaaataaaaaagatgattgatttacaaaatgctggatttgatataattatagttttggctgatttacacgcctatttaaaccagaaaggagagttggatgagattagaaaaataggagattataacaaaaaagtttttgaagcaatggggttaaaggcaaaatatgtttatggaagtgaattccagcttgataaggattatacactgaatgtctatagattggctttaaaaactaccttaaaaagagcaagaaggagtatggaacttatagcaagagaggatgaaaatccaaaggttgctgaagttatctatccaataatgcaggttaattatgatcattattatggcgttgatgttgcggttggagggatggagcagagaaaaatacacatgttagcaagggagcttttaccaaaaaaggttgtttgtattcacaaccctgtcttaacgggtttggatggagaaggaaagatgagttcttcaaaagggaattttatagctgttgatgactctccagaagagattagggctaagataaagaaagcatactgcccagctggagttgttgaaggaaatccaataatggagatagctaaatacttccttgaatatcctttaaccataaaagggccagaaaaatttggtggagatttgacagttaatagctatgaggagttagagagtttatttaaaaataaggaattgcatccaatgcgcttaaaaaatgctgtagctgaagaacttataaagattttagagccaattagaaagagattataataa

SEQ ID NO: 11 - DNA, 924 bp, Synthetic construct
  source 1..924: mol_type = other DNA
atggacgaatttgaaatgataaagagaaacacatctgaaattatcagcgaggaagagttaagagaggttttaaaaaaagatgaaaaatctgctgggatagggtttgaaccaagtggtaaaatacatttagggcattatctccaaataaaaaagatgattgatttacaaaatgctggatttgatataattatagttttggctgatttacacgcctatttaaaccagaaaggagagttggatgagattagaaaaataggagattataacaaaaaagtttttgaagcaatggggttaaaggcaaaatatgtttatggaagtgaattccagcttgataaggattatacactgaatgtctatagattggctttaaaaactaccttaaaaagagcaagaaggagtatggaacttatagcaagagaggatgaaaatccaaaggttgctgaagttatctatccaataatgcaggttaattgtgatcattatgggggcgttgatgttttggttggagggatggagcagagaaaaatacacatgttagcaagggagcttttaccaaaaaaggttgtttgtattcacaaccctgtcttaacgggtttggatggagaaggaaagatgagttcttcaaaagggaattttatagctgttgatgactctccagaagagattagggctaagataaagaaagcatactgcccagctggagttgttgaaggaaatccaataatggagatagctaaatacttccttgaatatcctttaaccataaaagggccagaaaaatttggtggagatttgacagttaatagctatgaggagttagagagtttatttaaaaataaggaattgcatccaatgcgcttaaaaaatgctgtagctgaagaacttataaagattttagagccaattagaaagagattataataa

SEQ ID NO: 12 - DNA, 924 bp, Synthetic construct
  source 1..924: mol_type = other DNA
atggacgaatttgaaatgataaagagaaacacatctgaaattatcagcgaggaagagttaagagaggttttaaaaaaagatgaaaaatctgctgggatagggtttgaaccaagtggtaaaatacatttagggcattatctccaaataaaaaagatgattgatttacaaaatgctggatttgatataattatagttttggctgatttacacgcctatttaaaccagaaaggagagttggatgagattagaaaaataggagattataacaaaaaagtttttgaagcaatggggttaaaggcaaaatatgtttatggaagtgaattccagcttgataaggattatacactgaatgtctatagattggctttaaaaactaccttaaaaagagcaagaaggagtatggaacttatagcaagagaggatgaaaatccaaaggttgctgaagttatctatccaataatgcaggttaatatttcgcattatgatggcgttgatgttttggttggagggatggagcagagaaaaatacacatgttagcaagggagcttttaccaaaaaaggttgtttgtattcacaaccctgtcttaacgggtttggatggagaaggaaagatgagttcttcaaaagggaattttatagctgttgatgactctccagaagagattagggctaagataaagaaagcatactgcccagctggagttgttgaaggaaatccaataatggagatagctaaatacttccttgaatatcctttaaccataaaagggccagaaaaatttggtggagatttgacagttaatagctatgaggagttagagagtttatttaaaaataaggaattgcatccaatgcgcttaaaaaatgctgtagctgaagaacttataaagattttagagccaattagaaagagattataataa

SEQ ID NO: 13 - protein, 239 aa, Synthetic construct
SKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGS

SEQ ID NO: 14 - protein, 248 aa, Synthetic construct
  MOD_RES 150: note = (2S)-2-amino-3-[4-[(E)-(1,3,5-trimethylpyrazol-4-yl )azo]-phenyl]propanoic acid hydrochloride
  MOD_RES 181: note = (2S)-2-amino-3-[4-[(E)-(1,3,5-trimethylpyrazol-4-yl )azo]-phenyl]propanoic acid hydrochloride
SKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVXIMADKQKNGIKVNFKIRHNIEDGSVQLADHXQQNTPIGDGPVLLPDNHYLSTQSKLSKDPNEKRDHMVLLEFVTAAGITLGMDELYKGSSHHHHHHGG

SEQ ID NO: 15 - protein, 160 aa, Synthetic construct
  MOD_RES 19: note = (2S)-2-amino-3-[4-[(E)-(1,3,5-trimethylpyrazol-4-yl )azo]-phenyl]propanoic acid hydrochloride
  MOD_RES 34: note = (2S)-2-amino-3-[4-[(E)-(1,3,5-trimethylpyrazol-4-yl )azo]-phenyl]propanoic acid hydrochloride
  MOD_RES 49: note = (2S)-2-amino-3-[4-[(E)-(1,3,5-trimethylpyrazol-4-yl )azo]-phenyl]propanoic acid hydrochloride
  MOD_RES 64: note = (2S)-2-amino-3-[4-[(E)-(1,3,5-trimethylpyrazol-4-yl )azo]-phenyl]propanoic acid hydrochloride
  MOD_RES 79: note = (2S)-2-amino-3-[4-[(E)-(1,3,5-trimethylpyrazol-4-yl )azo]-phenyl]propanoic acid hydrochloride
  MOD_RES 94: note = (2S)-2-amino-3-[4-[(E)-(1,3,5-trimethylpyrazol-4-yl )azo]-phenyl]propanoic acid hydrochloride
  MOD_RES 109: note = (2S)-2-amino-3-[4-[(E)-(1,3,5-trimethylpyrazol-4-yl )azo]-phenyl]propanoic acid hydrochloride
  MOD_RES 124: note = (2S)-2-amino-3-[4-[(E)-(1,3,5-trimethylpyrazol-4-yl )azo]-phenyl]propanoic acid hydrochloride
  MOD_RES 139: note = (2S)-2-amino-3-[4-[(E)-(1,3,5-trimethylpyrazol-4-yl )azo]-phenyl]propanoic acid hydrochloride
  MOD_RES 154: note = (2S)-2-amino-3-[4-[(E)-(1,3,5-trimethylpyrazol-4-yl )azo]-phenyl]propanoic acid hydrochloride
SKGPGVPGGGVPGAGVPGXGVPGGGVPGAGVPGXGVPGGGVPGAGVPGXGVPGGGVPGAGVPGXGVPGGGVPGAGVPGXGVPGGGVPGAGVPGXGVPGGGVPGAGVPGXGVPGGGVPGAGVPGXGVPGGGVPGAGVPGXGVPGGGVPGAGVPGXGPGGGG

SEQ ID NO: 16 - protein, 323 aa, Synthetic construct
  MOD_RES 145: note = (2S)-2-amino-3-[4-[(E)-(1,3,5-trimethylpyrazol-4-yl )azo]-phenyl]propanoic acid hydrochloride
  MOD_RES 155: note = (2S)-2-amino-3-[4-[(E)-(1,3,5-trimethylpyrazol-4-yl )azo]-phenyl]propanoic acid hydrochloride
  MOD_RES 305: note = (2S)-2-amino-3-[4-[(E)-(1,3,5-trimethylpyrazol-4-yl )azo]-phenyl]propanoic acid hydrochloride
  MOD_RES 315: note = (2S)-2-amino-3-[4-[(E)-(1,3,5-trimethylpyrazol-4-yl )azo]-phenyl]propanoic acid hydrochloride
GVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGXGVPGAGVPGXGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGGGVPGAGVPGXGVPGAGVPGXGVPGAGGY

SEQ ID NO: 17 - protein, 303 aa, Synthetic construct
  MOD_RES 50: note = (2S)-2-amino-3-[4-[(E)-(1,3,5-trimethylpyrazol-4-yl )azo]-phenyl]propanoic acid hydrochloride
  MOD_RES 100: note = (2S)-2-amino-3-[4-[(E)-(1,3,5-trimethylpyrazol-4-yl )azo]-phenyl]propanoic acid hydrochloride
  MOD_RES 150: note = (2S)-2-amino-3-[4-[(E)-(1,3,5-trimethylpyrazol-4-yl )azo]-phenyl]propanoic acid hydrochloride
  MOD_RES 200: note = (2S)-2-amino-3-[4-[(E)-(1,3,5-trimethylpyrazol-4-yl )azo]-phenyl]propanoic acid hydrochloride
  MOD_RES 250: note = (2S)-2-amino-3-[4-[(E)-(1,3,5-trimethylpyrazol-4-yl )azo]-phenyl]propanoic acid hydrochloride
  MOD_RES 300: note = (2S)-2-amino-3-[4-[(E)-(1,3,5-trimethylpyrazol-4-yl )azo]-phenyl]propanoic acid hydrochloride
GVPGGGVPGAGVPGGGVPGAGVPGGGVPGAGVPGGGVPGAGVPGGGVPGXGVPGGGVPGAGVPGGGVPGAGVPGGGVPGAGVPGGGVPGAGVPGGGVPGXGVPGGGVPGAGVPGGGVPGAGVPGGGVPGAGVPGGGVPGAGVPGGGVPGXGVPGGGVPGAGVPGGGVPGAGVPGGGVPGAGVPGGGVPGAGVPGGGVPGXGVPGGGVPGAGVPGGGVPGAGVPGGGVPGAGVPGGGVPGAGVPGGGVPGXGVPGGGVPGAGVPGGGVPGAGVPGGGVPGAGVPGGGVPGAGVPGGGVPGXGGY

SEQ ID NO: 18 - protein, 303 aa, Synthetic construct
  MOD_RES 30: note = (2S)-2-amino-3-[4-[(E)-(1,3,5-trimethylpyrazol-4-yl )azo]-phenyl]propanoic acid hydrochloride
  MOD_RES 60: note = (2S)-2-amino-3-[4-[(E)-(1,3,5-trimethylpyrazol-4-yl )azo]-phenyl]propanoic acid hydrochloride
  MOD_RES 90: note = (2S)-2-amino-3-[4-[(E)-(1,3,5-trimethylpyrazol-4-yl )azo]-phenyl]propanoic acid hydrochloride
  MOD_RES 120: note = (2S)-2-amino-3-[4-[(E)-(1,3,5-trimethylpyrazol-4-yl )azo]-phenyl]propanoic acid hydrochloride
  MOD_RES 150: note = (2S)-2-amino-3-[4-[(E)-(1,3,5-trimethylpyrazol-4-yl )azo]-phenyl]propanoic acid hydrochloride
  MOD_RES 180: note = (2S)-2-amino-3-[4-[(E)-(1,3,5-trimethylpyrazol-4-yl )azo]-phenyl]propanoic acid hydrochloride
  MOD_RES 210: note = (2S)-2-amino-3-[4-[(E)-(1,3,5-trimethylpyrazol-4-yl )azo]-phenyl]propanoic acid hydrochloride
  MOD_RES 240: note = (2S)-2-amino-3-[4-[(E)-(1,3,5-trimethylpyrazol-4-yl )azo]-phenyl]propanoic acid hydrochloride
  MOD_RES 270: note = (2S)-2-amino-3-[4-[(E)-(1,3,5-trimethylpyrazol-4-yl )azo]-phenyl]propanoic acid hydrochloride
  MOD_RES 300: note = (2S)-2-amino-3-[4-[(E)-(1,3,5-trimethylpyrazol-4-yl )azo]-phenyl]propanoic acid hydrochloride
GVPGGGVPGAGVPGGGVPGAGVPGGGVPGXGVPGGGVPGAGVPGGGVPGAGVPGGGVPGXGVPGGGVPGAGVPGGGVPGAGVPGGGVPGXGVPGGGVPGAGVPGGGVPGAGVPGGGVPGXGVPGGGVPGAGVPGGGVPGAGVPGGGVPGXGVPGGGVPGAGVPGGGVPGAGVPGGGVPGXGVPGGGVPGAGVPGGGVPGAGVPGGGVPGXGVPGGGVPGAGVPGGGVPGAGVPGGGVPGXGVPGGGVPGAGVPGGGVPGAGVPGGGVPGXGVPGGGVPGAGVPGGGVPGAGVPGGGVPGXGGY

SEQ ID NO: 19 - protein, 324 aa, Synthetic construct
  MOD_RES 26: note = (2S)-2-amino-3-[4-[(E)-(1,3,5-trimethylpyrazol-4-yl )azo]-phenyl]propanoic acid hydrochloride
  MOD_RES 58: note = (2S)-2-amino-3-[4-[(E)-(1,3,5-trimethylpyrazol-4-yl )azo]-phenyl]propanoic acid hydrochloride
  MOD_RES 90: note = (2S)-2-amino-3-[4-[(E)-(1,3,5-trimethylpyrazol-4-yl )azo]-phenyl]propanoic acid hydrochloride
  MOD_RES 122: note = (2S)-2-amino-3-[4-[(E)-(1,3,5-trimethylpyrazol-4-yl )azo]-phenyl]propanoic acid hydrochloride
  MOD_RES 154: note = (2S)-2-amino-3-[4-[(E)-(1,3,5-trimethylpyrazol-4-yl )azo]-phenyl]propanoic acid hydrochloride
  MOD_RES 186: note = (2S)-2-amino-3-[4-[(E)-(1,3,5-trimethylpyrazol-4-yl )azo]-phenyl]propanoic acid hydrochloride
  MOD_RES 218: note = (2S)-2-amino-3-[4-[(E)-(1,3,5-trimethylpyrazol-4-yl )azo]-phenyl]propanoic acid hydrochloride
  MOD_RES 250: note = (2S)-2-amino-3-[4-[(E)-(1,3,5-trimethylpyrazol-4-yl )azo]-phenyl]propanoic acid hydrochloride
  MOD_RES 282: note = (2S)-2-amino-3-[4-[(E)-(1,3,5-trimethylpyrazol-4-yl )azo]-phenyl]propanoic acid hydrochloride
  MOD_RES 314: note = (2S)-2-amino-3-[4-[(E)-(1,3,5-trimethylpyrazol-4-yl )azo]-phenyl]propanoic acid hydrochloride
GVPGAGVPGGGVPGAGKVPGGGVPGXGVPGGGKVPGAGVPGGGVPGAGKVPGGGVPGXGVPGGGKVPGAGVPGGGVPGAGKVPGGGVPGXGVPGGGKVPGAGVPGGGVPGAGKVPGGGVPGXGVPGGGKVPGAGVPGGGVPGAGKVPGGGVPGXGVPGGGKVPGAGVPGGGVPGAGKVPGGGVPGXGVPGGGKVPGAGVPGGGVPGAGKVPGGGVPGXGVPGGGKVPGAGVPGGGVPGAGKVPGGGVPGXGVPGGGKVPGAGVPGGGVPGAGKVPGGGVPGXGVPGGGKVPGAGVPGGGVPGAGKVPGGGVPGXGVPGGGKGGY

SEQ ID NO: 20 - protein, 402 aa, Synthetic construct
  MOD_RES 62: note = (2S)-2-amino-3-[4-[(E)-(1,3,5-trimethylpyrazol-4-yl )azo]-phenyl]propanoic acid hydrochloride
  MOD_RES 110: note = (2S)-2-amino-3-[4-[(E)-(1,3,5-trimethylpyrazol-4-yl )azo]-phenyl]propanoic acid hydrochloride
  MOD_RES 158: note = (2S)-2-amino-3-[4-[(E)-(1,3,5-trimethylpyrazol-4-yl )azo]-phenyl]propanoic acid hydrochloride
  MOD_RES 254: note = (2S)-2-amino-3-[4-[(E)-(1,3,5-trimethylpyrazol-4-yl )azo]-phenyl]propanoic acid hydrochloride
  MOD_RES 302: note = (2S)-2-amino-3-[4-[(E)-(1,3,5-trimethylpyrazol-4-yl )azo]-phenyl]propanoic acid hydrochloride
  MOD_RES 350: note = (2S)-2-amino-3-[4-[(E)-(1,3,5-trimethylpyrazol-4-yl )azo]-phenyl]propanoic acid hydrochloride
MGHHHHHHGHHHHHHGRGDSPYSGRGDSPYSGRGDSPYSGRGDSPYSGRGDSPYSGRGDSPXSGRGDSPYSGRGDSPYSGRGDSPYSGRGDSPYSGRGDSPYSGRGDSPXSGRGDSPYSGRGDSPYSGRGDSPYSGRGDSPYSGRGDSPYSGRGDSPXSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPXSGRGDSPYSGRGDSPYSGRGDSPYSGRGDSPYSGRGDSPYSGRGDSPXSGRGDSPYSGRGDSPYSGRGDSPYSGRGDSPYSGRGDSPYSGRGDSPXSGRGDSPYSGRGDSPYSGRGDSPYSGRGDSPYSGRGDSPYSGRGDSPYSGGY

SEQ ID NO: 21 - DNA, 90 bp, Synthetic construct
  source 1..90: mol_type = other DNA
aagagttaagagaggttttaaaaaaagatgaaaaatctgctnnkatannktttgaaccaagtggtaaaatacatttagggcattatctcc

SEQ ID NO: 22 - DNA, 90 bp, Synthetic construct
  source 1..90: mol_type = other DNA
agatgattgatttacaaaatgctggatttgatataattatannkttgnnkgatttacacgcctatttaaaccagaaaggagagttggatg

SEQ ID NO: 23 - DNA, 90 bp, Synthetic construct
  source 1..90: mol_type = other DNA
tttttgaagcaatggggttaaaggcaaaatatgtttatggaagtnnknnknnkcttgataaggattatacactgaatgtctatagattgg

SEQ ID NO: 24 - DNA, 90 bp, Synthetic construct
  source 1..90: mol_type = other DNA
aaaagagcaagaaggagtatggaacttatagcaagagaggatgaaaatccaaaggttgctgaagttatcnnkccaataatgcaggttaat

SEQ ID NO: 25 - DNA, 90 bp, Synthetic construct
  source 1..90: mol_type = other DNA
ccaataatgcaggttaatnnknnkcattatnnkggcgttgatgttnnkgttggagggatggagcagagaaaaatacacatgttagcaagg

SEQ ID NO: 26 - protein, 16 aa, Synthetic construct
  MOD_RES 9: note = (2S)-2-amino-3-[4-[(E)-(1,3,5-trimethylpyrazol-4-yl )azo]-phenyl]propanoic acid hydrochloride
VPGGGVPGZGVPGGGK

SEQ ID NO: 27 - protein, 16 aa, Synthetic construct
  MOD_RES 9: note = (2S)-2-amino-3-[4-[(E)-(1,3,5-trimethylpyrazol-4-yl )azo]-phenyl]propanoic acid hydrochloride
VPGGGVPGYGVPGGGK

SEQ ID NO: 28 - protein, 16 aa, Synthetic construct
  MOD_RES 9: note = (2S)-2-amino-3-[4-[(E)-(1,3,5-trimethylpyrazol-4-yl )azo]-phenyl]propanoic acid hydrochloride
VPGGGVPGEGVPGGGK

(19) United States
(12) Patent Application Publication
(10) Pub. No.: US 2026/0218158 A1
(43) Pub. Date: Jul. 30, 2026

(54) MUTANT AMINOACYL TRNA SYNTHETASE, COMPOSITIONS COMPRISING SAME, AND USE THEREOF

(71) Applicant: B. G. NEGEV TECHNOLOGIES AND APPLICATIONS LTD., AT BEN-GURION UNIVERSITY, Beer-Sheva (IL)

(72) Inventors: Miriam AMIRAM, Beer Sheva (IL); Daniela STRUGACH, Beer Sheva (IL)

(73) Assignee: B. G. NEGEV TECHNOLOGIES AND APPLICATIONS LTD., AT BEN-GURION UNIVERSITY, Beer-Sheva (IL)

(21) Appl. No.: 19/140,276

(22) PCT Filed: Jan. 2, 2024
(86) PCT No.: PCT/IL2024/050005

§ 371 Date: Jun. 17, 2025

Related U.S. Application Data
(60) Provisional application No. 63/436,646, filed on Jan. 2, 2023.

(51) Int. Cl. C12N 9/00; C12N 13/00

(52) U.S. Cl. CPC C12N 9/93; C12N 13/00

(57) ABSTRACT

The present invention provides a protein including the amino acid sequence of SEQ ID NO: 7, wherein isoleucine at position 159 is substituted with Ser or Asp. Further provided are composition comprising the protein disclosed herein, as well as methods of using same, such as for producing a protein of interest including a non-standard amino acid.

### CROSS-REFERENCE TO RELATED APPLICATIONS

This application claims the benefit of priority of U.S. Provisional Patent Application No. 63/436,646, titled “MUTANT AMINOACYL TRNA SYNTHETASE, COMPOSITIONS COMPRISING SAME, AND USE THEREOF”, filed 2 Jan. 2023, the contents of which are incorporated herein by reference in their entirety.

### REFERENCE TO AN ELECTRONIC SEQUENCE LISTING

The contents of the electronic sequence listing (BGU-P-0129-PCT.xml; size: 57,640 bytes; and date of creation: Dec. 13, 2023) is herein incorporated by reference in its entirety.

### FIELD OF INVENTION

The present invention is directed to mutated forms of aminoacyl tRNA synthetase (aaRS), compositions comprising same, and use thereof.

### BACKGROUND OF THE INVENTION

The development of a versatile and robust technology to produce light-responsive proteins and protein-based polymers (PBPs) can facilitate the design of countless new user-controlled biological agents. In turn, these proteins and PBPs can be manipulated by light in space and time to elucidate or manipulate complex cellular behavior, activate or release a therapeutic on demand, and precisely control the growth and differentiation of cells and tissues. Although genetic fusion with natural or engineered light-responsive domains can be employed for such purposes, the relatively large fusion protein may interfere with the target protein's function or limit the types of functionality that can be manipulated with light. Alternatively, photo-switchable small molecules can be chemically conjugated to a reactive amino acid side chain or a peptide backbone. However, this methodology can be limited due to the efficiency of the conjugation chemistries, the functionalization of only accessible sites, or the chemical synthesis of small peptides. In contrast, engineering the protein translation machinery for incorporating photo-switchable unnatural amino acids (uAAs) can enable their precise installment at multiple defined positions within the protein chain and hence the synthesis, evolution, and optimization of light-responsive protein and PBP behaviors.

The most commonly used photo-switchable group is the azobenzene, where the basic molecule 1 (FIG. 1A) undergoes trans to cis isomerization in response to UV light, but can be red-shifted by installing various substituents on the aromatic rings. Indeed, the current inventors and others have recently described the genetic incorporation of several azobenzene derivatives as unnatural amino acids (uAAs) in proteins and PBPs. Nevertheless, several other newly described photo-switchable molecules, in which one of the benzene rings is replaced with five-membered heterocycles, have demonstrated superior properties for bi-directional photo-control, such as high photostationary state (PSS) compositions resulting in more complete photo-switching, and improved thermal stability resulting in long-lived cis-isomers. Specifically, arylazopyrazole 2 (FIG. 1B) displays near complete photo-switching in both directions and excellent thermal stability of the cis isomer.

There is still a great need for enzymes capable of incorporating an uAA including arylazopyrazole into a tRNA, such that a light-responsive protein harboring the photo-switchable arylazopyrazole.

### SUMMARY OF THE INVENTION

Herein described is the evolution of the first orthogonal aminoacyl-tRNA synthetase (aaRS) capable of efficient genetic incorporation of an arylazopyrazole-bearing uAA (AAP). Further, the inventors utilized the selected aaRS variants to genetically encode light-responsive protein-based polymers based on elastin- and resilin-like polypeptides (ELPs and RLPs, respectively). The present invention, in some embodiments, is based, at least in part, on the findings provided herein describing the photo-switchable properties of arylazopyrazole-decorated ELPs and RLPs. The present invention, in some embodiments, is further based, at least in part, in the results showing that arylazopyrazole incorporation generates superior light-responsive properties as compared with similar, azobenzene-decorated ELPs. All of the above, relies or stems from production of aaRS mutants, harboring specific and superior activity in the conjugation or pairing of an uAA comprising arylazopyrazole and a receptive tRNA, and use of same.

According to one aspect, there is provided a protein comprising the amino acid sequence of SEQ ID NO: 7, wherein isoleucine at position 159 (1159) is substituted with Ser or Asp.

According to another aspect, there is provided a polynucleotide encoding the protein disclosed herein.

According to another aspect, there is provided an artificial or synthetic nucleic acid molecule comprising the polynucleotide disclosed herein.

According to another aspect, there is provided a cell comprising any one of: (a) the protein disclosed herein; (b) the polynucleotide disclosed herein; (c) the artificial or synthetic nucleic acid molecule disclosed herein; and (d) any combination of (a) to (c).

According to another aspect, there is provided a composition comprising any one of: (a) the protein disclosed herein; (b) the polynucleotide disclosed herein; (c) the artificial or synthetic nucleic acid molecule disclosed herein; (d) the cell disclosed herein; and any combination of (a) to (d), and an acceptable carrier.

According to another aspect, there is provided method for producing a protein of interest (POI) comprising a nsAA, the method comprising culturing a cell comprising a polynucleotide encoding the POI and comprising a premature stop codon, under conditions sufficient for expression of the polynucleotide, wherein the cell comprises the protein disclosed herein, thereby producing the POI comprising a nsAA.

In some embodiments, the protein further comprises a substitution in at least one position selected from the group consisting of: 158, 162, 167, and any combination thereof, of SEQ ID NO: 7, wherein any one of: aspartate at position 158 (D158) is substituted with Cys, Met, Gly, Tyr, or Ile, leucin at position 162 (L162) is substituted with Gly, Tyr, or Asp, alanine at position 167 (A167) is substituted with Leu, Thr, or Tyr, and any combination thereof.

In some embodiments, the protein comprises I159 being substituted with Asp (I159D).

In some embodiments, the protein comprises any one of: D158 being substituted with Cys (D158C), L162 being substituted with Gly (L162G), A167 being substituted with Leu (A167L), and any combination thereof.

In some embodiments, the protein comprises D158C, I159D, L162G, and A167L.

In some embodiments, the protein further comprises a substitution in at least one position selected from the group consisting of: 32, 65, 257, 286, of SEQ ID NO: 7, wherein any one of: tyrosine at position 32 (Y32) being substituted with Gly, leucin at position (L65) being substituted with Val, arginine at position (R257) being substituted with Gly, aspartate at position 286 (D286) being substituted with Arg, and any combination thereof.

In some embodiments, the protein comprises an amino acid sequence having not more than 98% sequence homology or identity to SEQ ID NO: 7.

In some embodiments, the protein comprises an amino acid sequence set forth in any one of: SEQ ID Nos: 1-5, or a functional analog thereof having at least 95% homology thereto.

In some embodiments, the protein comprises an amino acid sequence set forth in SEQ ID NO: 4.

In some embodiments, the protein is characterized by being capable of pairing a non-standard amino acid (nsAA) with a tRNA.

In some embodiments, the nsAA comprises a photo-switchable group.

In some embodiments, the photo-switchable group is characterized by the structure:

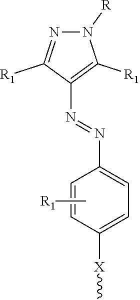

wherein R independently represents an optional substituted alkyl or H; wherein R1 represents one or more substituents, wherein each of the one or more substituents is independently selected from the group consisting of C1-C6 alkyl, halo, NO2, CN, OH, NH2, carbonyl, CONH2, CONR′2, CNNR2, CSNR2, CONH—OH, CONH—NH2, NHCOR′,NHCSR′, NHCNR′, —NC(═O)OR′, —NC(═O)NR′, —NC(═S)OR′, —NC(═S)NR′, SO2R′, SOR′, —SR′, SO2OR′, SO2N(R′)2, —NHNR′2, —NNR′, NH(C1-C6 alkyl), N(C1-C6 alkyl)2, C1-C6 alkoxy, C1-C6 haloalkoxy, hydroxy(C1-C6 alkyl), hydroxy(C1-C6 alkoxy), alkoxy(C1-C6 alkyl), alkoxy(C1-C6 alkoxy), amino(C1-C6 alkyl), CONH(C1-C6 alkyl), CON(C1-C6 alkyl)2, CO2H, CO2R′, —OCOR′, —OCOR′, —OC(═O)OR′, —OC(═O)NR′, —OC(═S)OR′, —OC(═S)NR′, a heteroatom, a cyclyl, an optionally substituted cycloalkyl, optionally substituted heterocyclyl, and a combination thereof, wherein each R′ is independently selected from hydrogen, alkyl, cycloalkyl, alkenyl, aryl, heteroaryl, optionally bonded through a ring carbon, or through a heteroatom) or heterocyclyl, optionally bonded through a ring carbon, or through a heteroatom); wherein X is a bond or an alkyl; and wherein a wavy bond represents an attachment point to a backbone of the nsAA.

In some embodiments, the nsAA comprises arylazopyrazole (AAP).

In some embodiments, the tRNA comprises an anticodon complementary to a stop codon.

In some embodiments, the stop codon is an amber codon (UAG).

In some embodiments, the artificial or synthetic nucleic acid molecule is an expression vector or a plasmid.

In some embodiments, the cell is a bacterial cell.

In some embodiments, the composition further comprises a tRNA comprising an anticodon complementary to a stop codon, a nsAA, or both.

In some embodiments, the composition further comprises a tRNA comprising an anticodon complementary to a stop codon paired to a nsAA.

In some embodiments, culturing comprises supplementing the cell with an effective amount of the nsAA.

In some embodiments, producing comprises increasing production levels of the POI comprising the nsAA, compared to a control.

In some embodiments, increasing is by at least 1.5-fold compared to the control.

In some embodiments, the method further comprises a step comprising subjecting the produced POI comprising the nsAA to light at a wavelength configured to induce conformational switch of the photo-switchable group.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1B include structural illustrations of (1A) the azobenzene-uAA (AB) and azobenzene photo-isomerization, and (1B) the arylazopyrazole-uAA (AAP) and arylazopyrazole photo-isomerization. PSS compositions and half-lives indicated by Weston et al., (J. Am. Chem. Soc., 2014) and Bandara and Burdette (Chem. Soc. Rev., 2012).

FIGS. 2A-2F include vertical bar graphs and three dimensional (3D) structures. (2A) Production of GFP (2TAG) by AzoRS-4, which was the progenitor enzyme used to generate the library, and five evolved AAPRS variants of the invention, expressed from a single chromosomal copy. (2B) Production of GFP (2TAG) by AzoRS-4 (SEQ ID NO: 6), which was the progenitor enzyme used to generate the library, and five evolved AAPRS (SEQ ID Nos: 1-5) variants of the invention, expressed from a multi-copy plasmid. (2C) Production of ELP-GFP fusion proteins containing 10 instances of the AAP-uAA depicted in FIG. 1B, and expressed by episomal versions of AzoRS-4, and the herein disclosed evolved variants, AAPRS1-4, in the C321.ΔRF1 strain. The level of GFP fluorescence indicates the production of the ELP-GFP fusion and, therefore, the efficiency of uAA incorporation. (2D-2F) A comparison of the AA-binding pocket of native MjTyrRS (2D) compared with AzoRS-4 (2E), and AAPRS-4 (2F). The structural models were generated (based on PDB ID: 1J1U) using the PyMOL program (Schrödinger, LLC). n=3; error bars indicate s.d.

FIGS. 3A-3F include graphs showing the characterization of the light-responsive properties of ELPs containing multiple instances of AAP. (3A-3C) Turbidity profiles as a function of temperature and light irradiation for ELPs (25 μM solutions in water) containing either 10 (3A), 6 (3B) or 2 (3C, supplemented with 2 M NaCl), instances of AAP after 30 min irradiation with each wavelength. (3D) Turbidity profiles as a function of temperature and light irradiation for ELP containing 10 instances of AAP after 1 min irradiation with each wavelength. (3E) Turbidity profile as a function of temperature and light irradiation of ELP₆₀(Gly/AAP×10) (25 μM solutions in water, except for ELP60(AAP×2), which was supplemented with 2 M NaCl). (3F) Reversibility of the light-mediated transition over ten cycles of 3- or 1-min illumination of ELPs (25 μM solutions in water) containing 10 instances of AAP at 38° C. Absorbance at 600 nm is normalized to that of the dark-adapted sample.

FIG. 4 includes vertical bar graphs showing incorporation of 10 instances of AAP in a single ELP by high-performance aaRSs previously described by the current inventors and expressed from a multi-copy plasmid in C321.ΔRF1. Error bars indicate SD (n=3).

FIG. 5 includes a vertical bar graph showing incorporation of 10 instances of AB in a single ELP by variants of the invention (SEQ ID Nos: 1-5) being selected for AAP incorporation and expressed from a multi-copy plasmid in C321.ΔRF1. Error bars indicate SD (n=3).

FIGS. 6A-6D include graphs showing ultraviolet-visible (UV-vis) spectra of dark-adapted or irradiated (30 min): (6A) AAP (250 μM), (6B) ELP₆₀(AAP×10), (6C) ELP₆₀(AAP×6), and (6D) ELP₆₀(AAP×2), each at 25 μM in water.

FIGS. 7A-7C include graphs showing ΔLCST_{cis/trans} of ELP₆₀(AAP×6) as a function of NaCl concentration in water (7A), and supplemented with 1 M (7B), and 2 M NaCl (7C).

FIGS. 8A-8D include graphs showing UV-vis spectra of AAP (250 μM; (8A-8B)), and ELP₆₀(AAP×10; 25 μM; (8C-8D)) as a function of irradiation wavelength and time.

### DETAILED DESCRIPTION OF THE INVENTION

The present invention, in some embodiments, is directed to a protein comprising the amino acid sequence set forth in SEQ ID NO: 7, wherein isoleucine at position 159 (I159) is substituted with serine (Ser) or aspartic acid (Asp).

In some embodiments, the protein is an aminoacyl tRNA synthetase (aaRS). In some embodiments, the protein is an aminoacyl tRNA synthetase (aaRS) mutant. In some embodiments, the protein is an aminoacyl tRNA synthetase (aaRS) mutant of SEQ ID NO: 7. In some embodiments, the protein of the invention comprises an amino acid sequence set forth in any one of SEQ ID Nos: 1-5, or a functional analog thereof.

In some embodiments, the protein or aaRS mutant, as disclosed herein, is capable of conjugating or pairing an unnatural amino acid comprising arylazopyrazole (arylazopyrazole-uAA (AAP)) and a receptive tRNA.

In some embodiments, protein or aaRS mutant, as disclosed herein is characterized by having superior or increased activity of conjugation or pairing of an unnatural amino acid comprising arylazopyrazole (arylazopyrazole-uAA (AAP)) and a receptive tRNA, compared to a control. In some embodiments, a control is or comprises a control protein or a control aaRS.

In some embodiments, a control comprises a wildtype aaRS. In some embodiments, a wildtype aaRS comprises an amino acid sequence as set forth in SEQ ID NO: 7. In some embodiments, a control comprises a protein comprising an amino acid sequence set forth in SEQ ID NO: 7. In some embodiments, a control comprises a protein comprising SEQ ID NO: 6.

According to some embodiments, there is provided a protein, comprising an amino acid sequence comprising a substitution in position 159 of SEQ ID NO: 7, wherein the isoleucine residue corresponding to 1159 is substituted with Ser or Asp.

In some embodiments, the protein, further comprises at least one additional substitution in at least one position selected from: 158, 162, 167, or any combination thereof, of SEQ ID NO: 7. In some embodiments, the protein further comprises at least a second mutation in at least one position selected from: 158, 162, 167, or any combination thereof, of SEQ ID NO: 7.

In some embodiments, the at least one additional mutation is a substitution mutation, as disclosed herein.

In some embodiments, the aspartate residue corresponding to D158 is substituted with Cys, Met, Gly, Tyr, or Ile.

In some embodiments, the leucin residue corresponding to L162 is substituted with Gly, Tyr, or Asp.

In some embodiments, the alanine residue corresponding to A167 is substituted with Leu, Thr, or Tyr.

In some embodiments, the aspartate residue corresponding to D158 is substituted with Cys, Met, Gly, Tyr, or Ile, and the leucin residue corresponding to L162 is substituted with Gly, Tyr, or Asp.

In some embodiments, the aspartate residue corresponding to D158 is substituted with Cys, Met, Gly, Tyr, or Ile, and the alanine residue corresponding to A167 is substituted with Leu, Thr, or Tyr, and any combination thereof.

In some embodiments, the leucin residue corresponding to L162 is substituted with Gly, Tyr, or Asp, the alanine residue corresponding to A167 is substituted with Leu, Thr, or Tyr, and any combination thereof.

In some embodiments, the aspartate residue corresponding to D158 is substituted with Cys, Met, Gly, Tyr, or Ile, the leucin residue corresponding to L162 is substituted with Gly, Tyr, or Asp, and the alanine residue corresponding to A167 is substituted with Leu, Thr, or Tyr.

In some embodiments, the protein comprises I159 being substituted with Asp (I159D).

In some embodiments, the protein comprises D158 being substituted with Cys (D158C), and L162 being substituted with Gly (L162G).

In some embodiments, the protein comprises D158 being substituted with Cys (D158C), and A167 being substituted with Leu (A167L).

In some embodiments, the protein comprises L162 being substituted with Gly (L162G), and A167 being substituted with Leu (A167L).

In some embodiments, the protein comprises D158 being substituted with Cys (D158C), L162 being substituted with Gly (L162G), and A167 being substituted with Leu (A167L).

In some embodiments, the protein comprises D158C, I159D, L162G, and A167L.

In some embodiments, the protein further comprises at least a second substitution. In some embodiments, protein further comprises at least a third mutation.

In some embodiments, the protein further comprises at least the second substitution or the at least third mutation is in at least one position selected from: 32, 65, 257, 286, of SEQ ID NO: 1.

In some embodiments, the tyrosine residue corresponding to Y32 being substituted with Gly, and the leucin residue corresponding to L65 being substituted with Val.

In some embodiments, the tyrosine residue corresponding to Y32 being substituted with Gly, and the arginine corresponding to R257 being substituted with Gly.

In some embodiments, the tyrosine residue corresponding to Y32 being substituted with Gly, and the aspartate corresponding to D286 being substituted with Arg.

In some embodiments, the leucin residue corresponding to L65 being substituted with Val, and the arginine corresponding to R257 being substituted with Gly.

In some embodiments, the leucin residue corresponding to L65 being substituted with Val, and the aspartate corresponding to D286 being substituted with Arg, and any combination thereof.

In some embodiments, the arginine corresponding to R257 being substituted with Gly, and the aspartate corresponding to D286 being substituted with Arg.

In some embodiments, the tyrosine residue corresponding to Y32 being substituted with Gly, the leucin residue corresponding to L65 being substituted with Val, the arginine corresponding to R257 being substituted with Gly, and the aspartate corresponding to D286 being substituted with Arg.

In some embodiments, the protein comprises an amino acid sequence having not more than 95%, 96%, 97.5%, or 98% sequence homology or identity to SEQ ID NO: 6 or SEQ ID NO: 7, or any value and range therebetween. Each possibility represents a separate embodiment of the invention.

In some embodiments, the protein comprises an amino acid sequence having at least 97.5%, 98%, 99% or 100% sequence homology or identity to SEQ ID Nos: 1-5, or any value and range therebetween. Each possibility represents a separate embodiment of the invention.

In some embodiments, a wildtype aaRS comprises the amino acid sequence:

(SEQ ID NO: 7)
MDEFEMIKRNTSEIISEEELREVLKKDEKSAYIGFEPSGKIHLGHYLQI
KKMIDLQNAGFDIIILLADLHAYLNQKGELDEIRKIGDYNKKVFEAMGL
KAKYVYGSEFQLDKDYTLNVYRLALKTTLKRARRSMELIAREDENPKVA
EVIYPIMQVNDIHYLGVDVAVGGMEQRKIHMLARELLPKKVVCIHNPVL
TGLDGEGKMSSSKGNFIAVDDSPEEIRAKIKKAYCPAGVVEGNPIMEIA
KYFLEYPLTIKRPEKFGGDLTVNSYEELESLFKNKELHPMDLKNAVAEE
LIKILEPIRKRL.

In some embodiments, the protein comprises the amino acid sequence set forth in any one of: SEQ ID Nos: 1-5.

In some embodiments, the protein of the invention comprises the amino acid sequence: MDEFEMIKRNTSEIISEEELREVLKKDEKSAGIGFEPSGKIHLGHYLQIKKMIDL QNAGFDIIIVLADLHAYLNQKGELDEIRKIGDYNKKVFEAMGLKAKYVYGSEF QLDKDYTLNVYRLALKTTLKRARRSMELIAREDENPKVAEVIYPIMQVNMDH YYGVDVTVGGMEQRKIHMLARELLPKKVVCIHNPVLTGLDGEGKMSSSKGNF IAVDDSPEEIRAKIKKAYCPAGVVEGNPIMEIAKYFLEYPLTIKGPEKFGGDLTV NSYEELESLFKNKELHPMRLKNAVAEELIKILEPIRKRL (SEQ ID NO: 1), or a functional analog thereof, having at least 95%, 96%, 97%, 98%, 99% or 100% sequence homology or identity thereto.

In some embodiments, the protein of the invention comprises the amino acid sequence: MDEFEMIKRNTSEIISEEELREVLKKDEKSAGIGFEPSGKIHLGHYLQIKKMIDL QNAGFDIIIVLADLHAYLNQKGELDEIRKIGDYNKKVFEAMGLKAKYVYGSEF QLDKDYTLNVYRLALKTTLKRARRSMELIAREDENPKVAEVIYPIMQVNGSH YYGVDVYVGGMEQRKIHMLARELLPKKVVCIHNPVLTGLDGEGKMSSSKGN FIAVDDSPEEIRAKIKKAYCPAGVVEGNPIMEIAKYFLEYPLTIKGPEKFGGDLT VNSYEELESLFKNKELHPMRLKNAVAEELIKILEPIRKRL (SEQ ID NO: 2), or a functional analog thereof, having at least 95%, 96%, 97%, 98%, 99% or 100% sequence homology or identity thereto.

In some embodiments, the protein of the invention comprises the amino acid sequence: MDEFEMIKRNTSEIISEEELREVLKKDEKSAGIGFEPSGKIHLGHYLQIKKMIDL QNAGFDIIIVLADLHAYLNQKGELDEIRKIGDYNKKVFEAMGLKAKYVYGSEF QLDKDYTLNVYRLALKTTLKRARRSMELIAREDENPKVAEVIYPIMQVNYDH YYGVDVAVGGMEQRKIHMLARELLPKKVVCIHNPVLTGLDGEGKMSSSKGN FIAVDDSPEEIRAKIKKAYCPAGVVEGNPIMEIAKYFLEYPLTIKGPEKFGGDLT VNSYEELESLFKNKELHPMRLKNAVAEELIKILEPIRKRL (SEQ ID NO: 3), or a functional analog thereof, having at least 95%, 96%, 97%, 98%, 99% or 100% sequence homology or identity thereto.

In some embodiments, the protein of the invention comprises the amino acid sequence: MDEFEMIKRNTSEIISEEELREVLKKDEKSAGIGFEPSGKIHLGHYLQIKKMIDL QNAGFDIIIVLADLHAYLNQKGELDEIRKIGDYNKKVFEAMGLKAKYVYGSEF QLDKDYTLNVYRLALKTTLKRARRSMELIAREDENPKVAEVIYPIMQVNCDH YGGVDVLVGGMEQRKIHMLARELLPKKVVCIHNPVLTGLDGEGKMSSSKGNF IAVDDSPEEIRAKIKKAYCPAGVVEGNPIMEIAKYFLEYPLTIKGPEKFGGDLTV NSYEELESLFKNKELHPMRLKNAVAEELIKILEPIRKRL (SEQ ID NO: 4), or a functional analog thereof, having at least 95%, 96%, 97%, 98%, 99% or 100% sequence homology or identity thereto.

In some embodiments, the protein of the invention comprises the amino acid sequence: MDEFEMIKRNTSEIISEEELREVLKKDEKSAGIGFEPSGKIHLGHYLQIKKMIDL QNAGFDIIIVLADLHAYLNQKGELDEIRKIGDYNKKVFEAMGLKAKYVYGSEF QLDKDYTLNVYRLALKTTLKRARRSMELIAREDENPKVAEVIYPIMQVNISHY DGVDVLVGGMEQRKIHMLARELLPKKVVCIHNPVLTGLDGEGKMSSSKGNFI AVDDSPEEIRAKIKKAYCPAGVVEGNPIMEIAKYFLEYPLTIKGPEKFGGDLTV NSYEELESLFKNKELHPMRLKNAVAEELIKILEPIRKRL (SEQ ID NO: 5), or a functional analog thereof, having at least 95%, 96%, 97%, 98%, 99% or 100% sequence homology or identity thereto.

The term “functional analog” as used herein, refers to a polypeptide or a protein that is similar, but not identical, to the protein of the invention, that still is capable of conjugating or pairing conjugating or pairing an unnatural amino acid comprising arylazopyrazole (arylazopyrazole-uAA (AAP)) and a receptive tRNA. A functional analog may have deletions or mutations that result in an amino acids sequence that is different than the amino acid sequence of the protein of the invention. It should be understood that all functional analogs of the protein of the invention would still be capable of conjugating or pairing an unnatural amino acid comprising AAP-uAA and a receptive tRNA. Further, a functional analog may be analogous to a fragment of the protein of the invention. In some embodiments, a fragment comprises at least 50, 70, 90, 120, 150, 175, 200, 220, 250, 270, 290, or 300 consecutive amino acids of the protein of the invention, or any value and range therebetween. Each possibility represents a separate embodiment of the invention.

As used herein, the term “analog” includes any peptide having an amino acid sequence substantially identical to one of the sequences specifically shown herein in which one or more residues have been conservatively substituted with a functionally similar residue and which displays the abilities as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. Each possibility represents a separate embodiment of the present invention.

As used herein, the terms “peptide”, “polypeptide” and “protein” are used interchangeably to refer to a polymer of amino acid residues. In another embodiment, the terms “peptide”, “polypeptide”, and “protein” as used herein encompass native peptides, peptidomimetics (typically including non-peptide bonds or other synthetic modifications) and the peptide analogues peptoids and semipeptoids or any combination thereof. In one embodiment, the terms “peptide”, “polypeptide”, and “protein” apply to naturally occurring amino acid polymers. In another embodiment, the terms “peptide”, “polypeptide”, and “protein” apply to amino acid polymers in which one or more amino acid residue is an artificial chemical analogue of a corresponding naturally occurring amino acid.

In some embodiments, the protein of the invention is characterized by being capable of or comprises the ability of pairing a non-standard amino acid (nsAA) or unnatural amino acid (uAA) with a tRNA.

As used herein, the terms “unnatural amino acid (uAA)” and non-standard amino acid (nsAA) refer to any amino acid, modified amino acid, or amino acid analog other than selenocysteine and the following twenty genetically encoded alpha-amino acids (e.g., “canonical”): alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

The terms “nsAA” and “uAA”, as used herein, are interchangeably.

In some embodiments, nsAA comprises a photo-switchable group.

As used herein, the term “photo-switchable group” refers to any group that is capable of undergoing or changing E/Z or trans/cis, respectively, configuration by photoisomerization using light. In some embodiments, light comprises visible light. In some embodiments, light comprises UV light. In some embodiments, light comprises visible light or UV light. In one embodiment, visible light comprises a wavelength of 520 nm. In one embodiment, UV light comprises a wavelength of 365 nm UV.

In some embodiments, a photo-switchable group is characterized by the structure:

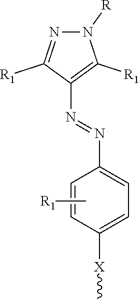

wherein R independently represents an optional substituted alkyl or H; wherein R1 represents one or more substituents, wherein each of the one or more substituents is independently selected from the group consisting of C1-C6 alkyl, halo, NO2, CN, OH, NH2, carbonyl, CONH2, CONR′2, CNNR2, CSNR2, CONH—OH, CONH—NH2, NHCOR′,NHCSR′, NHCNR′, —NC(═O)OR′, —NC(═O)NR′, —NC(═S)OR′, —NC(═S)NR′, SO2R′, SOR′, —SR′, SO2OR′, SO2N(R′)2, —NHNR′2, —NNR′, NH(C1-C6 alkyl), N(C1-C6 alkyl)2, C1-C6 alkoxy, C1-C6 haloalkoxy, hydroxy(C1-C6 alkyl), hydroxy(C1-C6 alkoxy), alkoxy(C1-C6 alkyl), alkoxy(C1-C6 alkoxy), amino(C1-C6 alkyl), CONH(C1-C6 alkyl), CON(C1-C6 alkyl)2, CO2H, CO2R′, —OCOR′, —OCOR′, —OC(═O)OR′, —OC(═O)NR′, —OC(═S)OR′, —OC(═S)NR′, a heteroatom, a cyclyl, an optionally substituted cycloalkyl, an optionally substituted heterocyclyl, and a combination thereof, wherein each R′ is independently selected from hydrogen, alkyl, cycloalkyl, alkenyl, aryl, heteroaryl (e.g., optionally bonded through a ring carbon, or through a heteroatom) or heterocyclyl (e.g., optionally bonded through a ring carbon, or through a heteroatom); wherein X is a bond or an alkyl; wherein a wavy bond represents an attachment point to a backbone of the nsAA.

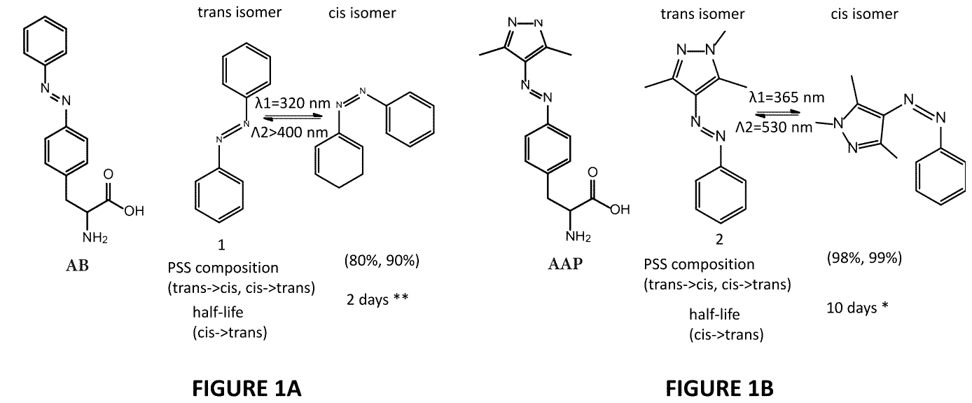

In some embodiments, a nsAA comprises arylazopyrazole (AAP), as disclosed herein (FIG. 1).

In some embodiments, tRNA comprises an anticodon complementary to a stop codon.

In some embodiments, a stop codon is selected from: an amber codon (UAG), ochre codon (UAA), or opal/umber codon (UGA).

In some embodiments, a stop codon is an amber codon (UAG).

In some embodiments, a tRNA comprises an anticodon selected from: CUA, UUA, or UCA.

In some embodiments, a tRNA comprises the anticodon CUA.

According to some embodiments, there is provided a polynucleotide encoding the protein of the invention, as disclosed herein.

As used herein, the terms “polynucleotide”, “polynucleotide sequence”, “nucleic acid sequence”, and “nucleic acid molecule” are used interchangeably herein. These terms encompass nucleotide sequences and the like. A polynucleotide may be a polymer of RNA or DNA that is single- or double-stranded, that optionally contains synthetic, non-natural or altered nucleotide bases.

In some embodiments, the polynucleotide comprises a nucleic acid sequence set forth in any one of SEQ ID Nos: 8-12.

In some embodiments, the polynucleotide comprises the nucleic acid sequence: ATGGACGAATTTGAAATGATAAAGAGAAACACATCTGAAATTATCAGCGA GGAAGAGTTAAGAGAGGTTTTAAAAAAAGATGAAAAATCTGCTGGGATAG GGTTTGAACCAAGTGGTAAAATACATTTAGGGCATTATCTCCAAATAAAAA AGATGATTGATTTACAAAATGCTGGATTTGATATAATTATAGTTTTGGCTG ATTTACACGCCTATTTAAACCAGAAAGGAGAGTTGGATGAGATTAGAAAA ATAGGAGATTATAACAAAAAAGTTTTTGAAGCAATGGGGTTAAAGGCAAA ATATGTTTATGGAAGTGAATTCCAGCTTGATAAGGATTATACACTGAATGT CTATAGATTGGCTTTAAAAACTACCTTAAAAAGAGCAAGAAGGAGTATGG AACTTATAGCAAGAGAGGATGAAAATCCAAAGGTTGCTGAAGTTATCTATC CAATAATGCAGGTTAATATGGATCATTATTATGGCGTTGATGTTACTGTTG GAGGGATGGAGCAGAGAAAAATACACATGTTAGCAAGGGAGCTTTTACCA AAAAAGGTTGTTTGTATTCACAACCCTGTCTTAACGGGTTTGGATGGAGAA GGAAAGATGAGTTCTTCAAAAGGGAATTTTATAGCTGTTGATGACTCTCCA GAAGAGATTAGGGCTAAGATAAAGAAAGCATACTGCCCAGCTGGAGTTGT TGAAGGAAATCCAATAATGGAGATAGCTAAATACTTCCTTGAATATCCTTT AACCATAAAAGGGCCAGAAAAATTTGGTGGAGATTTGACAGTTAATAGCT ATGAGGAGTTAGAGAGTTTATTTAAAAATAAGGAATTGCATCCAATGCGCT TAAAAAATGCTGTAGCTGAAGAACTTATAAAGATTTTAGAGCCAATTAGAA AGAGATTATAATAA (SEQ ID NO: 8), or an analog thereof having at least 80% sequence homology or identity thereto. In some embodiments, a polynucleotide comprising the nucleic acid sequence set forth in SEQ ID NO: 8 encodes a protein comprising the amino acid sequence set forth in SEQ ID NO: 1, as disclosed herein.

In some embodiments, the polynucleotide comprises the nucleic acid sequence: ATGGACGAATTTGAAATGATAAAGAGAAACACATCTGAAATTATCAGCGA GGAAGAGTTAAGAGAGGTTTTAAAAAAAGATGAAAAATCTGCTGGGATAG GGTTTGAACCAAGTGGTAAAATACATTTAGGGCATTATCTCCAAATAAAAA AGATGATTGATTTACAAAATGCTGGATTTGATATAATTATAGTTTTGGCTG ATTTACACGCCTATTTAAACCAGAAAGGAGAGTTGGATGAGATTAGAAAA ATAGGAGATTATAACAAAAAAGTTTTTGAAGCAATGGGGTTAAAGGCAAA ATATGTTTATGGAAGTGAATTCCAGCTTGATAAGGATTATACACTGAATGT CTATAGATTGGCTTTAAAAACTACCTTAAAAAGAGCAAGAAGGAGTATGG AACTTATAGCAAGAGAGGATGAAAATCCAAAGGTTGCTGAAGTTATCTATC CAATAATGCAGGTTAATGGGTCTCATTATTATGGCGTTGATGTTTATGTTGG AGGGATGGAGCAGAGAAAAATACACATGTTAGCAAGGGAGCTTTTACCAA AAAAGGTTGTTTGTATTCACAACCCTGTCTTAACGGGTTTGGATGGAGAAG GAAAGATGAGTTCTTCAAAAGGGAATTTTATAGCTGTTGATGACTCTCCAG AAGAGATTAGGGCTAAGATAAAGAAAGCATACTGCCCAGCTGGAGTTGTT GAAGGAAATCCAATAATGGAGATAGCTAAATACTTCCTTGAATATCCTTTA ACCATAAAAGGGCCAGAAAAATTTGGTGGAGATTTGACAGTTAATAGCTA TGAGGAGTTAGAGAGTTTATTTAAAAATAAGGAATTGCATCCAATGCGCTT AAAAAATGCTGTAGCTGAAGAACTTATAAAGATTTTAGAGCCAATTAGAA AGAGATTATAATAA (SEQ ID NO: 9), or an analog thereof having at least 80% sequence homology or identity thereto. In some embodiments, a polynucleotide comprising the nucleic acid sequence set forth in SEQ ID NO: 9 encodes a protein comprising the amino acid sequence set forth in SEQ ID NO: 2, as disclosed herein.

In some embodiments, the polynucleotide comprises the nucleic acid sequence: ATGGACGAATTTGAAATGATAAAGAGAAACACATCTGAAATTATCAGCGA GGAAGAGTTAAGAGAGGTTTTAAAAAAAGATGAAAAATCTGCTGGGATAG GGTTTGAACCAAGTGGTAAAATACATTTAGGGCATTATCTCCAAATAAAAA AGATGATTGATTTACAAAATGCTGGATTTGATATAATTATAGTTTTGGCTG ATTTACACGCCTATTTAAACCAGAAAGGAGAGTTGGATGAGATTAGAAAA ATAGGAGATTATAACAAAAAAGTTTTTGAAGCAATGGGGTTAAAGGCAAA ATATGTTTATGGAAGTGAATTCCAGCTTGATAAGGATTATACACTGAATGT CTATAGATTGGCTTTAAAAACTACCTTAAAAAGAGCAAGAAGGAGTATGG AACTTATAGCAAGAGAGGATGAAAATCCAAAGGTTGCTGAAGTTATCTATC CAATAATGCAGGTTAATTATGATCATTATTATGGCGTTGATGTTGCGGTTG GAGGGATGGAGCAGAGAAAAATACACATGTTAGCAAGGGAGCTTTTACCA AAAAAGGTTGTTTGTATTCACAACCCTGTCTTAACGGGTTTGGATGGAGAA GGAAAGATGAGTTCTTCAAAAGGGAATTTTATAGCTGTTGATGACTCTCCA GAAGAGATTAGGGCTAAGATAAAGAAAGCATACTGCCCAGCTGGAGTTGT TGAAGGAAATCCAATAATGGAGATAGCTAAATACTTCCTTGAATATCCTTT AACCATAAAAGGGCCAGAAAAATTTGGTGGAGATTTGACAGTTAATAGCT ATGAGGAGTTAGAGAGTTTATTTAAAAATAAGGAATTGCATCCAATGCGCT TAAAAAATGCTGTAGCTGAAGAACTTATAAAGATTTTAGAGCCAATTAGAA AGAGATTATAATAA (SEQ ID NO: 10), or an analog thereof having at least 80% sequence homology or identity thereto. In some embodiments, a polynucleotide comprising the nucleic acid sequence set forth in SEQ ID NO: 10 encodes a protein comprising the amino acid sequence set forth in SEQ ID NO: 3, as disclosed herein.

In some embodiments, the polynucleotide comprises the nucleic acid sequence: ATGGACGAATTTGAAATGATAAAGAGAAACACATCTGAAATTATCAGCGA GGAAGAGTTAAGAGAGGTTTTAAAAAAAGATGAAAAATCTGCTGGGATAG GGTTTGAACCAAGTGGTAAAATACATTTAGGGCATTATCTCCAAATAAAAA AGATGATTGATTTACAAAATGCTGGATTTGATATAATTATAGTTTTGGCTG ATTTACACGCCTATTTAAACCAGAAAGGAGAGTTGGATGAGATTAGAAAA ATAGGAGATTATAACAAAAAAGTTTTTGAAGCAATGGGGTTAAAGGCAAA ATATGTTTATGGAAGTGAATTCCAGCTTGATAAGGATTATACACTGAATGT CTATAGATTGGCTTTAAAAACTACCTTAAAAAGAGCAAGAAGGAGTATGG AACTTATAGCAAGAGAGGATGAAAATCCAAAGGTTGCTGAAGTTATCTATC CAATAATGCAGGTTAATTGTGATCATTATGGGGGCGTTGATGTTTTGGTTG GAGGGATGGAGCAGAGAAAAATACACATGTTAGCAAGGGAGCTTTTACCA AAAAAGGTTGTTTGTATTCACAACCCTGTCTTAACGGGTTTGGATGGAGAA GGAAAGATGAGTTCTTCAAAAGGGAATTTTATAGCTGTTGATGACTCTCCA GAAGAGATTAGGGCTAAGATAAAGAAAGCATACTGCCCAGCTGGAGTTGT TGAAGGAAATCCAATAATGGAGATAGCTAAATACTTCCTTGAATATCCTTT AACCATAAAAGGGCCAGAAAAATTTGGTGGAGATTTGACAGTTAATAGCT ATGAGGAGTTAGAGAGTTTATTTAAAAATAAGGAATTGCATCCAATGCGCT TAAAAAATGCTGTAGCTGAAGAACTTATAAAGATTTTAGAGCCAATTAGAA AGAGATTATAATAA (SEQ ID NO: 11), or an analog thereof having at least 80% sequence homology or identity thereto. In some embodiments, a polynucleotide comprising the nucleic acid sequence set forth in SEQ ID NO: 11 encodes a protein comprising the amino acid sequence set forth in SEQ ID NO: 4, as disclosed herein.

In some embodiments, the polynucleotide comprises the nucleic acid sequence: ATGGACGAATTTGAAATGATAAAGAGAAACACATCTGAAATTATCAGCGA GGAAGAGTTAAGAGAGGTTTTAAAAAAAGATGAAAAATCTGCTGGGATAG GGTTTGAACCAAGTGGTAAAATACATTTAGGGCATTATCTCCAAATAAAAA AGATGATTGATTTACAAAATGCTGGATTTGATATAATTATAGTTTTGGCTG ATTTACACGCCTATTTAAACCAGAAAGGAGAGTTGGATGAGATTAGAAAA ATAGGAGATTATAACAAAAAAGTTTTTGAAGCAATGGGGTTAAAGGCAAA ATATGTTTATGGAAGTGAATTCCAGCTTGATAAGGATTATACACTGAATGT CTATAGATTGGCTTTAAAAACTACCTTAAAAAGAGCAAGAAGGAGTATGG AACTTATAGCAAGAGAGGATGAAAATCCAAAGGTTGCTGAAGTTATCTATC CAATAATGCAGGTTAATATTTCGCATTATGATGGCGTTGATGTTTTGGTTGG AGGGATGGAGCAGAGAAAAATACACATGTTAGCAAGGGAGCTTTTACCAA AAAAGGTTGTTTGTATTCACAACCCTGTCTTAACGGGTTTGGATGGAGAAG GAAAGATGAGTTCTTCAAAAGGGAATTTTATAGCTGTTGATGACTCTCCAG AAGAGATTAGGGCTAAGATAAAGAAAGCATACTGCCCAGCTGGAGTTGTT GAAGGAAATCCAATAATGGAGATAGCTAAATACTTCCTTGAATATCCTTTA ACCATAAAAGGGCCAGAAAAATTTGGTGGAGATTTGACAGTTAATAGCTA TGAGGAGTTAGAGAGTTTATTTAAAAATAAGGAATTGCATCCAATGCGCTT AAAAAATGCTGTAGCTGAAGAACTTATAAAGATTTTAGAGCCAATTAGAA AGAGATTATAATAA (SEQ ID NO: 12), or an analog thereof having at least 80% sequence homology or identity thereto. In some embodiments, a polynucleotide comprising the nucleic acid sequence set forth in SEQ ID NO: 12 encodes a protein comprising the amino acid sequence set forth in SEQ ID NO: 8, as disclosed herein.

According to some embodiments, there is provided an artificial or synthetic nucleic acid molecule comprising the polynucleotide disclosed herein.

As used herein, the terms “artificial” or synthetic” refer to manmade product, e.g., nucleic acid molecules being generated artificially, such as by in vitro means, in a lab.

In some embodiments, the artificial or synthetic nucleic acid molecule is an expression vector or a plasmid.

The term “expression” as used herein refers to the biosynthesis of a gene product, including the transcription and/or translation of the gene product. Thus, expression of a nucleic acid molecule may refer to transcription of the nucleic acid fragment (e.g., transcription resulting in mRNA or other functional RNA) and/or translation of RNA into a precursor or mature protein (polypeptide).

Expressing of a gene within a cell is well known to one skilled in the art. It can be carried out by, among many methods, transfection, viral infection, or direct alteration of the cell's genome. In some embodiments, the gene is in an expression vector such as plasmid or viral vector. One such example of an expression vector containing p16-Ink4a is the mammalian expression vector pCMV p16 INK4A available from Addgene.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the gene is operably linked to a promoter. The term “operably linked” is intended to mean that the nucleotide sequence of interest is linked to the regulatory element or elements in a manner that allows for expression of the nucleotide sequence (e.g. in an in vitro transcription/translation system or in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

According to some embodiments, there is provided a cell comprising: (a) the mutated aaRS disclosed herein; (b) the polynucleotide disclosed herein; (c) the artificial or synthetic nucleic acid molecule disclosed herein; or (d) any combination of (a) to (c).

In some embodiments, the cell is a prokaryote cell. In some embodiments, the cell is a bacterial cell. In some embodiments, the bacterial cell is an E. coli cell. In some embodiments, the cell is a transformed, transgenic, or transduced cell. In some embodiments, the cell is genetically compatible for expression of the mutated aaRS disclosed herein. In some embodiments, a cell being genetically compatible for expression of the mutated aaRS disclosed herein, is characterized by being devoid of UAG or UGA or UAA termination function. In some embodiments, genetically compatible refers to a cell comprising a genome being devoid of UAG stop codon(s). In some embodiments, genetically compatible refers to a cell comprising a genome being devoid of UGA stop codon(s). In some embodiments, genetically compatible refers to a cell comprising a genome being devoid of UAA stop codon(s).

According to some embodiments, there is provided composition comprising any one of: (a) the mutated aaRS disclosed herein; (b) the polynucleotide disclosed herein; (c) the artificial or synthetic nucleic acid molecule disclosed herein; (d) the cell disclosed herein; or (e) any combination of (a) to (d), and an acceptable carrier.

In some embodiments, the composition further comprises a tRNA comprising an anticodon complementary to a stop codon, a nsAA, and both.

In some embodiments, the composition further comprises a tRNA comprising an anticodon complementary to a stop codon paired or conjugated to a nsAA.

In some embodiments, “paired” or “conjugated” is by a bond. In some embodiments, a bond is or comprises a covalent bond.

According to some embodiments, there is provided a method for producing a protein of interest (POI) comprising a nsAA, the method comprising culturing a cell comprising a polynucleotide encoding the POI and comprising a premature stop codon, wherein the cell comprises the protein of the invention, thereby producing the POI comprising a nsAA.

In some embodiments, the cell comprises the protein of the invention, a polynucleotide encoding thereof, or both.

In some embodiments, culturing is under conditions sufficient for expression of the polynucleotide encoding the POI.

Methods for culturing a cell are common and would be apparent to one of skill in the art.

In some embodiments, culturing comprises supplementing the cell with an effective amount of the nsAA.

In some embodiments, the method further comprises supplementing the cell with an effective amount of the nsAA.

In some embodiments, producing comprises increasing production levels of the POI comprising the nsAA compared to a control. In some embodiments, a control is or comprises a control cell.

In some embodiments, a control comprises a cell comprising the protein of the invention as disclosed herein or a polynucleotide encoding thereof, and being devoid of a nsAA, as disclosed herein. In some embodiments, a control comprises a cell contact only with a nsAA in the absence of the protein of the invention as disclosed herein. In some embodiments, a control comprises a cell contact with azobenzene-uAA (AB). In some embodiments, a control comprises a cell contact with an aaRS comprising the amino acid sequence set forth in SEQ ID NO: 6. In some embodiments, a control comprises a cell contact with an aaRS comprising the amino acid sequence set forth in SEQ ID NO: 7.

In some embodiments, increasing is by at least 1.5-fold, 2-fold, 4-fold, or 5-fold, compared to a control as disclosed herein, or aby value and range therebetween. Each possibility represents a separate embodiment of the invention.

In some embodiments, increasing is by 1.5-fold to 20-fold, 2-fold-100-fold, 4-fold-50-fold, or 5-fold-500-fold, compared to a control as disclosed herein. Each possibility represents a separate embodiment of the invention.

In some embodiments, the method further comprises a step comprising subjecting the produced POI comprising the nsAA to light or a light source.

In some embodiments, the step comprising subject the POI to light is proceeding the contacting step.

In some embodiments, subjecting is to a wavelength configured to induce conformational switch of the photo-switchable group.

In some embodiments, a wavelength configured to induce conformational switch of the photo-switchable group comprises: (i) 300-400 nm; (ii) 500-600 nm; or (iii) both (i) and (ii).

In some embodiments, a wavelength configured to induce conformational switch of the photo-switchable group comprises: (i) 330-380 nm; (ii) 510-550 nm; or (iii) both (i) and (ii).

In some embodiments, a wavelength configured to induce conformational switch of the photo-switchable group comprises: (i) 355-375 nm; (ii) 520-540 nm; or (iii) both (i) and (ii).

### General

Any concentration ranges, percentage range, or ratio range recited herein are to be understood to include concentrations, percentages or ratios of any integer within that range and fractions thereof, such as one tenth and one hundredth of an integer, unless otherwise indicated.

Any number range recited herein relating to any physical feature, such as polymer subunits, size or thickness, are to be understood to include any integer within the recited range, unless otherwise indicated.

As used herein, the terms “subject” or “individual” or “animal” or “patient” or “mammal,” refers to any subject, particularly a mammalian subject, for whom therapy is desired, for example, a human.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include”, and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Materials

(2S)-2-amino-3-[4-[(E)-(1,3,5-trimethylpyrazol-4-yl) azo]-phenyl]propanoic acid hydrochloride (AAP-uAA, 1) was purchased from Chiroblock. Restriction endonucleases and ligation enzymes were purchased from New England Biolabs. DNA amplification was performed using The KAPA2G Fast HotStart ReadyMix or the KAPA HiFi PCR kit (Roche). Plasmid purification was conducted with Plasmid HiYield mini-prep (RBC Bioscience) and the PCR/restriction product was purified using a HiYield gel/PCR extraction kit (RBC Bioscience). Ligation was performed using the Quick Ligation™ Kit or with the T4 DNA Ligase, both purchased from New England Biolabs. Ligation products were transformed into 5-alpha Competent E. coli (High Efficiency) or Stb12 Competent E. coli (High Efficiency), purchased from New England Biolabs. SDS solution was purchased from Bio-Rad. Anhydrotetracycline hydrochloride was purchased from Sigma-Aldrich. C321.AA (Isaacs lab) was a gift from Farren Isaacs (Addgene plasmids #73581). Isomerization experiments were performed with 365 nm (UV) and 530 nm (green) mounted LEDs (M365L3 and M530L3, Thor-Labs).

### Diversification and Selection of AzoRS Variants

aaRS libraries were generated by MAGE-based diversification of the previously isolated genomically integrated mutants AzoRS-4. Prior to MAGE cycling, cultures were established by inoculating the liquid medium with a single bacterial colony or by adding 30 μl of a confluent liquid culture (1:100 dilution) at 34° C. to mid-logarithmic growth phase bacterial cells (OD₆₀₀=0.6-0.7) in a shaking incubator. To induce the expression of the lambda-red recombination proteins, the cell cultures were shifted to 42° C. for 15 min and then immediately chilled on ice. Of these cultures, 1 ml of cells was centrifuged at 4° C. at 15,000 g for 30 s, the supernatant medium was removed, and the cells were resuspended in milli-Q water. Then, the cells were spun down, the supernatant was removed, and the washing procedure was repeated. After a final 30 s spin, the supernatant was removed and MAGE oligos (5-6 μM in DNase-free water) were added to the cell pellet. The oligo-cell mixture was transferred to a pre-chilled 1 mm gap electroporation cuvette (Bio-Rad) and electroporated under the following conditions: 1.8 kV, 200 V, and 25 mF. LB medium (3 ml) was immediately added to the electroporated cells, which were then recovered from electroporation and grown at 34° C. for 3-3.5 h. Once the cells reached the mid-log stage, they were used in additional MAGE cycles, subjected to negative and positive selection cycles, or frozen for further use.

After 5-10 rounds of diversification with MAGE and of negative (using varying concentrations of colicin E1 in the absence of 1) and positive (using varying concentrations of SDS in the presence of 1), and a final negative selection, the improved AzoRS variants were screened by plating cells on LB plates supplemented with appropriate antibiotics, L-Arabinose (0.2%), anhydrotetracycline (60 ng ml-1), and 1 (0.25 mM). Colonies expressing high levels of GFP were selected and subjected to further GFP expression analysis by intact-cell fluorescence measurements in the presence or absence of 1. The aaRS genes of the best-performing colonies were analyzed by Sanger sequencing.

### Plasmid Construction

Plasmids bearing the OTS variants for AAP-uAA incorporation were constructed by inserting aaRS genes into a previously described plasmid (pEvol) harboring a p15A origin of replication and a chloramphenicol resistance marker. The evolved genomic aaRS genes were PCR-amplified from chromosomal templates. All variants were inserted sequentially by using the flanking restriction sites BglII and SalI to obtain inducible expression under the control of the araBAD promoter and the rrnB terminator. The second constitutive copy of the aaRS typically found in the pEvol system was removed. Ligation was conducted with the Quick Ligation™ Kit (NEB®) and the ligation products were transformed into NEB® 5-alpha Competent E. coli (High Efficiency), later plated on LB-agar plates supplemented with chloramphenicol (25 μg ml⁻¹), and analyzed by Sanger sequencing.

### Analysis of GFP Expression by Intact-Cell Fluorescence Measurements

For 96-well plate-based assays, strains harboring chromosomally integrated orthogonal translation systems and GFP reporter plasmids were inoculated from frozen stocks and grown to confluence overnight. Cultures were then inoculated at a 1:50 dilution in 2×YT or LB medium supplemented with kanamycin (30 μg ml-1). For cells harboring the plasmid-based orthogonal translation system and GFP reporter proteins, the media were also supplemented with chloramphenicol (25 μg ml-1). Cells were allowed to grow at 34° C. to an OD₆₀₀ of 0.5-0.8 in a shaking plate incubator at 567 rpm (~3 h). The expression of aaRS was then induced by adding arabinose (0.2%); GFP expression was induced by adding anhydrotetracycline (60 ng ml-1); and the uAA was added at a concentration of 0.25 mM. Following expression, the cells were centrifuged at 4,000 g for 5 min, the supernatant medium was removed, and the cells were resuspended in PBS. GFP fluorescence was measured on a Biotek spectrophotometric plate reader by using excitation and emission wavelengths of 485 nm and 528 nm, respectively. Fluorescence signals were normalized by dividing the fluorescence counts by the OD₆₀₀ reading.

### ELP Expression and Purification

Before batch expression, starter cultures (1:40 v/v of final expression volume) of 2×YT media, supplemented with kanamycin (30 μg ml-1) and chloramphenicol (25 μg ml-1), were inoculated with transformed cells from either a fresh agar plate or from stocks stored at −80° C., incubated overnight at 34° C. while shaking at 220 rpm, and transferred to expression flasks containing 2×YT media, antibiotics, arabinose (0.2%), and AAP-uAA (0.25 mM). For the expression of ELP₆₀(10TAG), ELP₆₀(6TAG), and ELP₆₀(2TAG) by AzoRS-4, the C321.ΔRF1 strain, supplemented with AAP-uAA (0.25 mM) and arabinose (0.2%), was incubated at 34° C. for 4-5 h and then protein expression was induced with isopropyl β-d-1-thiogalactopyranoside (IPTG, 1 mM). The cells were harvested 24 h after inoculation by centrifugation at 4,000 g for 30 min at 4° C. The cell pellet was then resuspended by vortex in milli-Q water (~4 ml) and either stored at −80° C. or purified immediately. For purification, resuspended pellets were lysed by ultrasonic disruption (18 cycles of 10 s sonication, separated by 40 s intervals of rest). Poly(ethyleneimine) was added (0.2 ml of a 10% solution) to each lysed suspension before centrifugation at 4,000 g for 15 min at 4° C. to separate cell debris from the soluble cell lysate. All ELP constructs were purified by a modified inverse transition cycling (ITC) protocol consisting of multiple “hot” and “cold” spins by using sodium chloride to trigger the phase transition. Before purification, the soluble cell lysate was incubated for 1-2 min at 42-55° C. to denature the native E. coli proteins. The cell lysate was then cooled on ice, centrifuged for 2 min at ~14,000 rpm, and the pellet was discarded. For “hot” spins, the ELP phase transition was triggered by adding sodium chloride to the cell lysate or to the product of a previous cycle of ITC at a final concentration of ~5 M. The solutions were then centrifuged at ~14,000 rpm for 10 min and the pellets were resuspended in milli-Q water, after which a 2 min “cold” spin was performed without sodium chloride to remove denatured contaminant. Additional rounds of ITC were conducted as needed using a saturated solution of sodium chloride until sufficient purification was achieved.

Protein concentrations were calculated by measuring the OD₂₈₀ of the purified protein according to the following extinction coefficients: ELP₆₀(tyrosine×10): 16,390, ELP₆₀(1×10): 27,889, ELP₆₀(1×6): 17,669.4, and ELP₆₀(1×2): 7449.8, based on the extinction coefficient of AAP (2,554.9 M cm⁻¹).

### GRGDSPYS₄₀ Expression and Purification

Batch expression was performed as described above for ELP production. The cells were harvested 24 h after inoculation by centrifugation at 4,000 g for 30 min at 4° C. Purification was performed according to a previously described protocol.

Protein concentrations were calculated by measuring the OD₂₈₀ of the purified protein in 8M urea according to the following extinction coefficients: GRGDSPYS₄₀-(WT): 61,090, GRGDSPYS₄₀ (AAP×6): 67,989.4.

### Intact Mass Measurements

The intact mass was measured using MALDI-TOF/TOF autoflex speed at the Ilse Katz Institute for Nanoscale Science and Technology (Ben-Gurion University of the Negev). Spectrum analysis was performed by the Flexanalysis software.

### Phase Transition Analysis

To characterize the inverse transition temperature of ELP variants, the OD₆₀₀ of the ELP solution (in milli-Q water, unless otherwise noted) was monitored as a function of temperature, with heating and cooling performed at a rate of 1° C. min⁻¹ on a UV-vis spectrophotometer equipped with a multicell thermoelectric temperature controller (Thermo Scientific).

### Dynamic Light Scattering (DLS) Analysis

ELP self-assembly was analyzed using a Zetasizer Nano ZS (Malvern Pananalytical). For each sample, 12-17 acquisitions (determined automatically by the instrument) were obtained at 10° C. Populations comprising less than 1% of the total mass (by volume) were excluded from the analysis.

### Statistics

Statistical analyses were performed with GraphPad Prism software, with p<0.05 considered statistically significant. GFP production by each aaRS variant in the presence of AAP-uAAs was compared with GFP production by the progenitor system using ANOVA followed by Dunnett's post-hoc test, assuming a Gaussian distribution and unequal variability of differences.

### Example 1

### Evolving aaRSs for Multi-Site AAP-uAA Incorporation

The inventors began by determining the ability of a previously evolved aaRSs, derived from the Methanocaldococcus jannaschii tyrosyl-tRNA synthetase (MjTyrRS; SEQ ID NO: 7), for the incorporation of AAP (10 instances per protein) in the Escherichia coli strain C321.ΔRF1 which lacks all the native TAG codons and their associated release factor (RF-1). To this end, the inventors utilized a previously described ELP-based reporter protein ELP (10TAG)-GFP to indicate the multi-site incorporation of AAP in TAG codons (Table 1).

TABLE 1
GFP and protein-polymer sequences used in this study

| Protein | Amino acid sequence (* denotes the TAG codon) | SEQ ID NO: |
|---|---|---|
| GFP-WT | SKGEELFTGVVPILVELDGDVNGHKFSVRGEGEG | 13 |
| | DATNGKLTLKFICTTGKLPVPWPTLVTTLTYGVQ | |
| | CFSRYPDHMKRHDFFKSAMPEGYVQERTISFKDD | |
| | GTYKTRAEVKFEGDTLVNRIELKGIDFKEDGNILG | |
| | HKLEYNFNSHNVYITADKQKNGIKANFKIRHNVE | |
| | DGSVQLADHYQQNTPIGDGPVLLPDNHYLSTQSV | |
| | LSKDPNEKRDHMVLLEFVTAAGITHGMDELYKGS | |
| GFP(2TAG) | SKGEELFTGVVPILVELDGDVNGHKFSVSGEGEG | 14 |
| | DATYGKLTLKFICTTGKLPVPWPTLVTTLTYGVQ | |
| | CFSRYPDHMKQHDFFKSAMPEGYVQERTIFFKDD | |
| | GNYKTRAEVKFEGDTLVNRIELKGIDFKEDGNILG | |
| | HKLEYNYNSHNVXIMADKQKNGIKVNFKIRHNIE | |
| | DGSVQLADHXQQNTPIGDGPVLLPDNHYLSTQSK | |
| | LSKDPNEKRDHMVLLEFVTAAGITLGMDELYKGS | |
| | SHHHHHHGG | |
| ELP(10TAG)-GFP | SKGPG(VPGGGVPGAGVPGXG)₁₀PGGGG-(GFP-WT) | 15 |
| ELP₆₀(2TAG) | G[(VPGGGVPGAG)₁₄(VPGXGVPGAG)]₂GY | 16 |
| ELP₆₀(6TAG) | G[(VPGGGVPGAG)₄(VPGGGVPGXG)]₆GY | 17 |
| ELP60(10TAG) | G[(VPGGGVPGAG)₂(VPGGG VPGXG)]₁₀GY | 18 |
| ELP₆₀(10TAG)_{MS} | G[(VPGAGVPGGGVPGAG)K(VPGGGVPGXGVPGGG)K]₁₀GGY | 19 |
| GRGDSPYS₄₀(6TAG) | MGHHHHHHGHHHHHH(GRGDSPYSGRG DSPYS | 20 |
| | GRGDS PYSGRGDSPY SGRGDSPYSG RGDSPXS)₃(GRGDSPYSG | |
| | RGDSPYSGRG DSPYSGRGDS PYSG RGDSPY SGRGDSPYS)]₂GGY | |

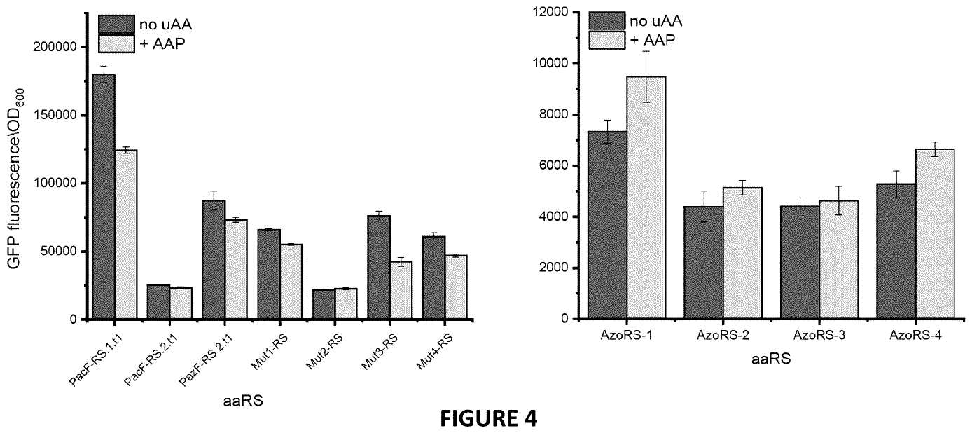

A GFP fluorescence assay indicated that variants previously described by the current inventors were incapable of multi-site incorporation of AAP, even when expressed from a multi-copy plasmid in C321.ΔRF1 (FIG. 4).

To enable the multi-site incorporation of azobenzene-uAAs, the inventors utilized a modified protein-evolution strategy that was previously developed by the current inventors to identify improved MjTyrRS mutants, which can efficiently charge an amber suppressor tRNA with AAP-uAA in C321.ΔRF1. Briefly, genomically integrated aaRS variants were subjected to 5-10 rounds of multiplex automated genome engineering (MAGE)-based diversification, followed by successive tolC-mediated negative-positive-negative selection cycles (ColE1-mediated negative selection or SDS-mediated positive selection). The first (negative) selection cycle was used to eliminate non-orthogonal variants generated in the diversification process, which, even if rare, would otherwise be enriched in the subsequent positive selection cycle; the second (positive) selection cycle was used to enrich the efficient aaRS variants; and the third (negative) selection cycle was used to eliminate “cheater” non-orthogonal clones generated in response to the stress applied in the positive selection step.

Given the similarity between AB and AAP, the inventors chose to perform the MAGE-based diversification on the genomically integrated AzoRS-4 which was previously evolved for high-efficiency incorporation of AB. Given a previous analysis made by the current inventors of the relevant AAs for diversification in the MjTyrRS uAA-binding pocket, the inventors selected 9 residues for diversification using degenerate ssDNA oligonucleotides (Table 2).

TABLE 2
Degenerate ssDNA MAGE oligonucleotides used in this study

| Targeted residues | | |
|---|---|---|
| in the uAA | | |
| binding pocket | Oligonucleotide sequence | SEQ ID NO: |
| L32, G34 | a*a*gagttaagagaggttttaaaaaaagatgaaaaatctgctnnkatan | 21 |
| | nktttgaaccaagtggtaaaatacatttagggcattatctcc | |
| L65, A67 | a*g*atgattgatttacaaaatgctggatttgatataattatannkttgnnk | 22 |
| | gatttacacgcctatttaaaccagaaaggagagttggatg | |
| E107, F108, Q109 | t*t*tttgaagcaatggggttaaaggcaaaatatgtttatggaagtnnknn | 23 |
| | knnkcttgataaggattatacactgaatgtctatagattgg | |
| Y151 | a*a*aagagcaagaaggagtatggaacttatagcaagagaggatgaaa | 24 |
| | atccaaaggttgctgaagttatcnnkccaataatgcaggttaat | |
| G158, C159, R162, | c*c*aataatgcaggttaatnnknnkcattatnnkggcgttgatgttnnk | 25 |
| A167 | gttggagggatggagcagagaaaaatacacatgttagcaagg | |

Single-stranded DNA oligonucleotides with two phosphorothioate bonds at the 5′ end (denoted
by *) were purchased from Integrated DNA Technologies. The degenerate base n represents all
four bases, and k represents G/T.

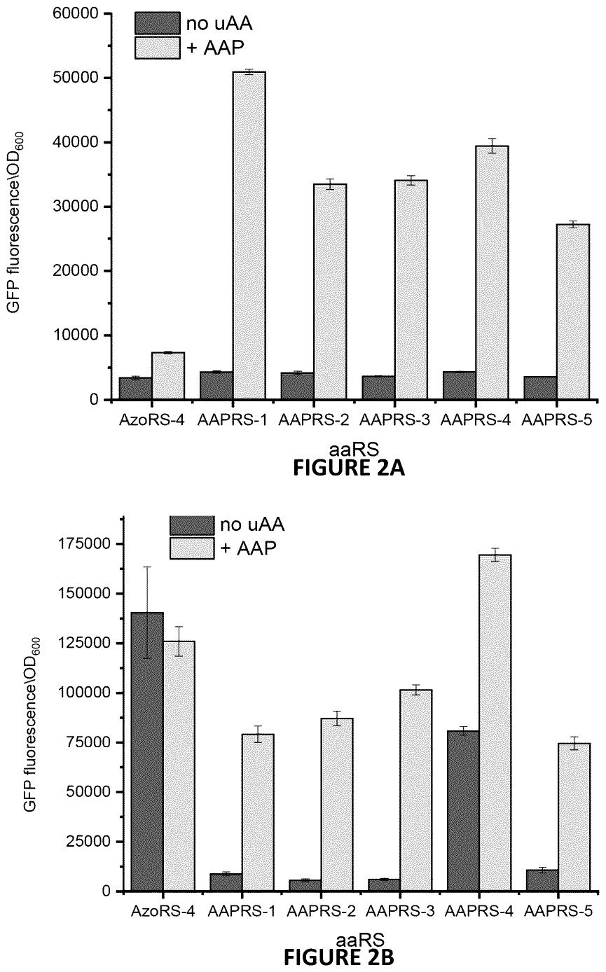

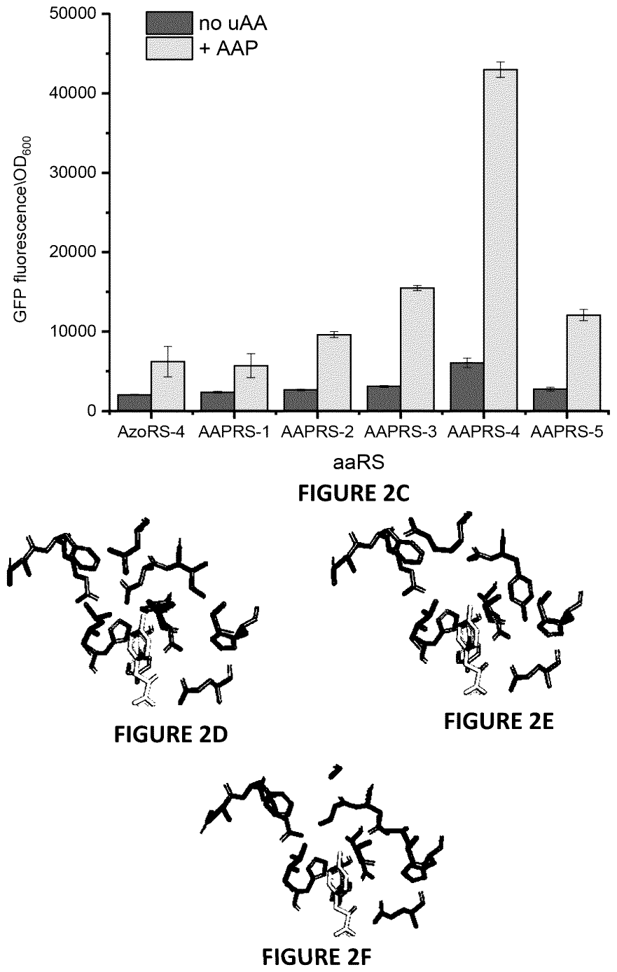

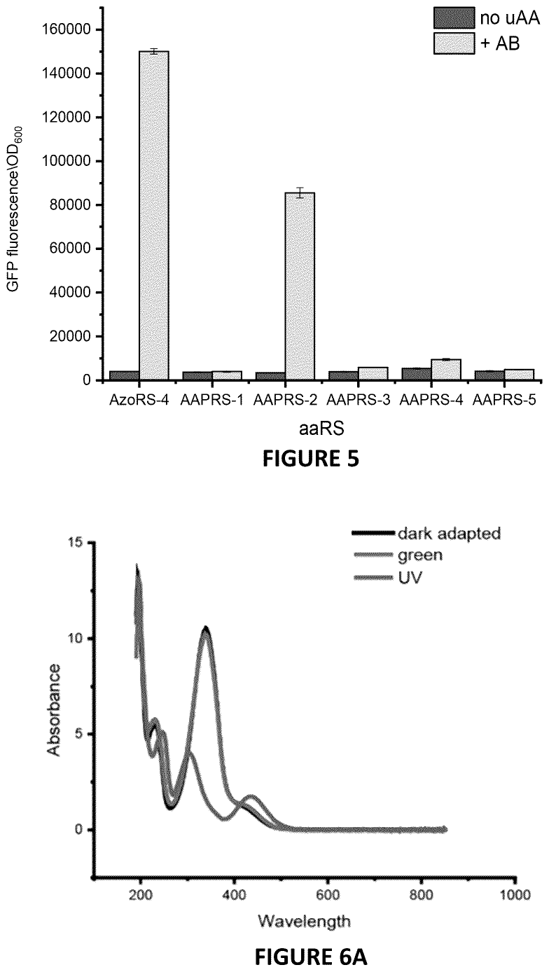

Following the diversification and selection steps, the production of GFP (2TAG) in the presence of AAP was used to evaluate activity in genomically integrated individual clones. This analysis revealed several variants that, when expressed from a single chromosomal copy, were capable of improved GFP (2TAG) production compared with the parent enzyme (FIG. 2A). After Sanger-sequencing of all the evolved variants (Table 3), the inventors evaluated them for the multi-site (2 or 10) incorporation of AAP by co-transforming C321.ΔRF1 with plasmids carrying the above-mentioned reporter proteins and episomal versions of each evolved variant. As expected, the expression of the evolved aaRSs from multi-copy plasmids increased their ability to incorporate multiple (2 or 10) instances of AAP. Notably, one of the evolved variants, designated AAPRS-4 (SEQ ID NO: 4), increased the expression of ELP (10TAG)-GFP containing AAP, by ~7-fold, as compared with the progenitor AzoRS-4 (FIGS. 2B-2C). Interestingly, as compared with the progenitor AzoRS-4 (SEQ ID NO: 6), all selected mutants harbored mutations in the same 4 amino acid sites targeted for mutagenesis, namely amino acids 158, 159, 162 and 167. Modelling of the mutations found in MjTyrRS, AzoRS-4 and AAPRS-4 suggests that mutagenesis in the above-mentioned sites somewhat decrease the size of the uAA binding pocket as compared to that formed in AzoRS-4 (FIGS. 2D-2F). Not surprisingly, all but one of the variants selected for AAP incorporation generate very low amounts of the ELP (10TAG)-GFP reporter protein in the presence of AB (FIG. 5) suggesting that mutually orthogonal AARSs may be utilized to simultaneously encode several photo-switchable uAAs.

TABLE 3
Annotations of specific mutations in evolved aaRS variants, as
compared with the WT Methanocaldococcus jannaschii tyrosyl-tRNA
synthetase (MjTyrRS) sequence and the AzoRS-4 sequence, which
was used to generate the library for selection of AAPRSS

| | SEQ | Position | | | | | | | | |
|---|---|---|---|---|---|---|---|---|---|---|
| Annotation | ID NO: | 2 | 5 | 107 | 108 | 109 | 158 | 159 | 162 | 167 |
| WT-MjTyrRS | 7 | Y | L | E | F | Q | D | I | L | A |
| AzoRS-4 | 6 | G | V | E | F | Q | G | Y | R | A |
| AAPRS-1 | 1 | G | V | E | F | Q | M | D | Y | T |
| AAPRS-2 | 2 | G | V | E | F | Q | G | S | Y | Y |
| AAPRS-3 | 3 | G | V | E | F | Q | Y | D | R | A |
| AAPRS-4 | 4 | G | V | E | F | Q | C | D | G | L |
| AAPRS-5 | 5 | G | V | E | F | Q | I | S | D | L |

In addition to the indicated mutations, all mutants also harbor the R257G and D286R mutations, which have been shown to improve tRNA binding.

### Example 2

### Genetically Encoding Phase-Separation Behavior in ELPs

To examine the effect of the photo-isomerization of AAP on the phase-separation behavior of ELPs, the inventors utilized a set of ELP variants previously designed by the current inventors to analyze the effect of AAP incorporation on the ELP Tt. The ELP pentapeptides are composed of glycine and alanine amino acids alternating in the X-guest residue position, with 2, 6 or 10 TAG codons (for the incorporation of AAP) distributed equally along the guest residue of the ELP [termed ELP₆₀(2TAG), ELP₆₀(6TAG), and ELP₆₀(10TAG), respectively, Table 1). The inventors selected this set of hydrophilic ELPs as hosts for AAP incorporation since similarly to AB and other aromatic uAAs, the hydrophobic AAP molecule was expected to reduce the Tt when incorporated in multiple sites in the ELPs. Throughout the paper, proteins expressed from the above-mentioned ELP genes are named according to the number and identity of the amino acid incorporated in the TAG codons. For example, ELP₆₀(AAP×10) is the protein product of the ELP₆₀(10TAG) gene, wherein AAP was incorporated in 10 encoded TAG codons.

The inventors first produced the ELP₆₀(AAP×10) protein in the C321.ΔRF1 strain by using AAPRS-4. To determine protein yields, the inventors purified small batches of ELP₆₀(AAP×10), where the protein yields were 35.69±3.69, as compared with ~40 mg L⁻¹ of ELP₆₀(WT) (previously reported). The inventors evaluated the accuracy of incorporating AAP by analyzing tryptic fragments of the MS-optimized reporter protein ELP₆₀(10TAG)_{MS} (Table 1) expressed with AAP (ELP₆₀(AAP×10)_{MS}) by liquid chromatography-MS (LC-MS), which identifies and assesses the extent of natural amino acid misincorporation. The incorporation of AAP by AAPRS-4 was detected in ~97% of the total ions (Table 4).

TABLE 4
Sequence and signal intensities of peptides identified LC-MS of tryptic
fragments. Z denoted the AAP-uAA. ELP₆₀(10x1)_{MS}, expressed by
AAPRS-4 in the C321.ARF1 strain

| | | Instances | | | |
|---|---|---|---|---|---|
| | SEQ | of peptide | % of | MH+ | |
| Sequence | ID NO: | sequenced | peptides | [Da] | XCorr |
| VPGGGVPGZGVPGGGK | 26 | 142 | 97% | 1474.791 | 3.93 |
| VPGGGVPGYGVPGGGK | 27 | 3 | 2% | 1354.711 | 3.82 |
| VPGGGVPGEGVPGGGK | 28 | 1 | 1% | 1320.691 | 2.11 |

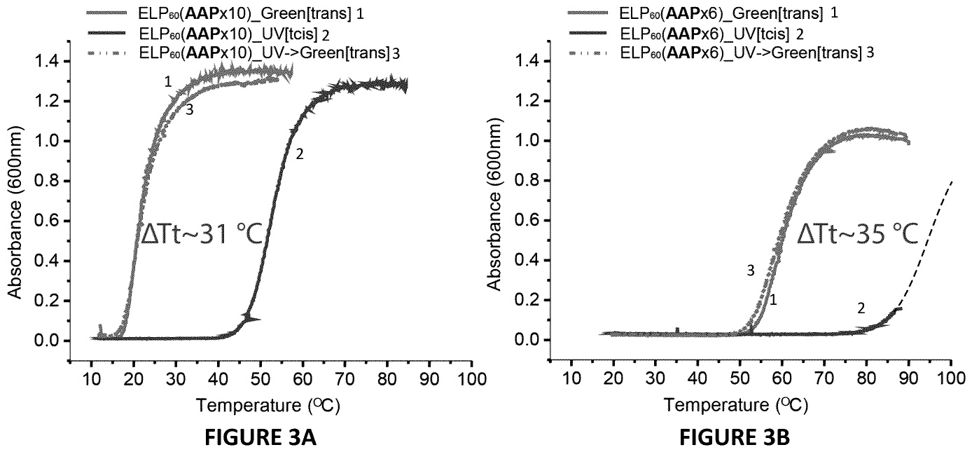

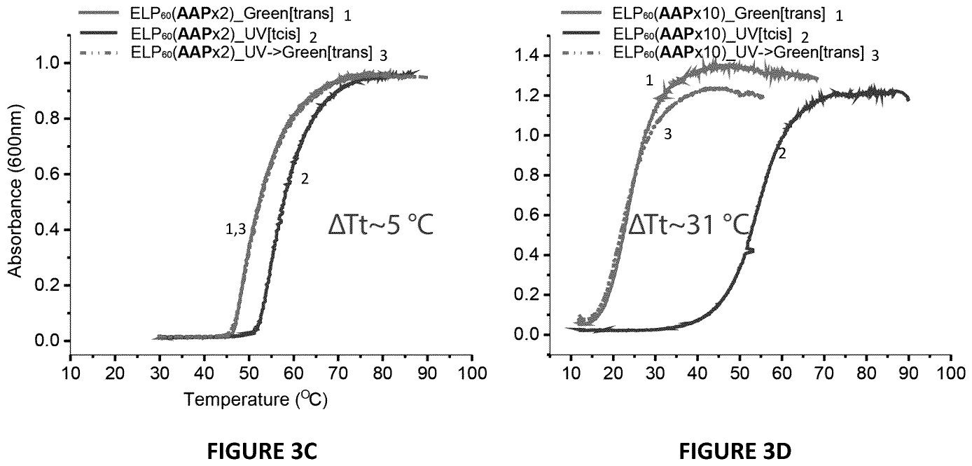

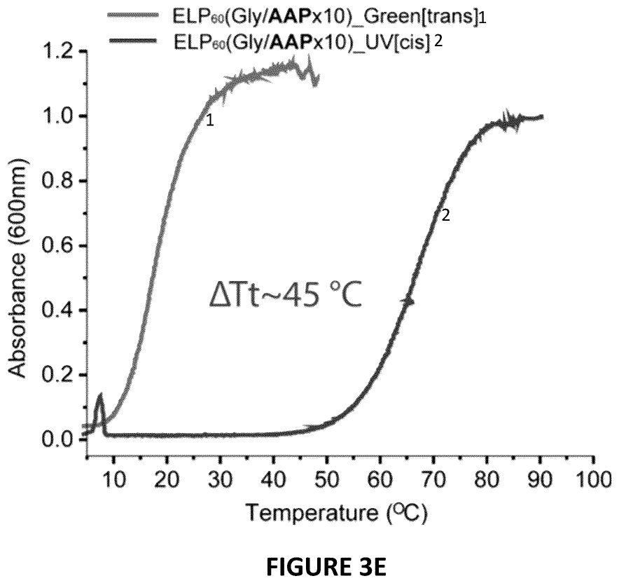

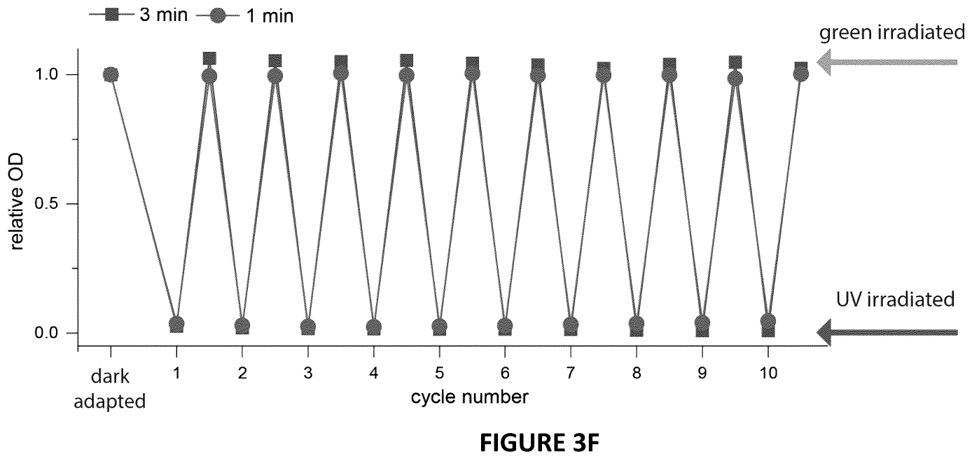

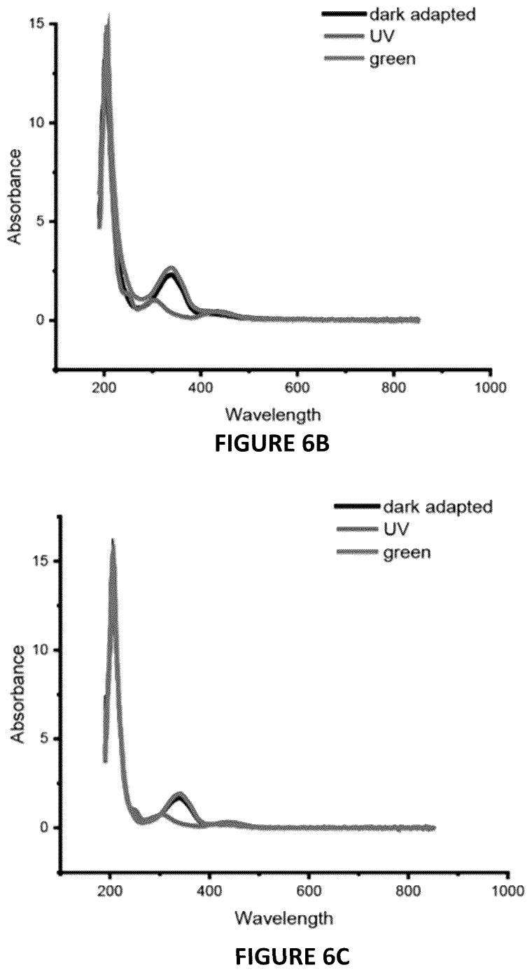

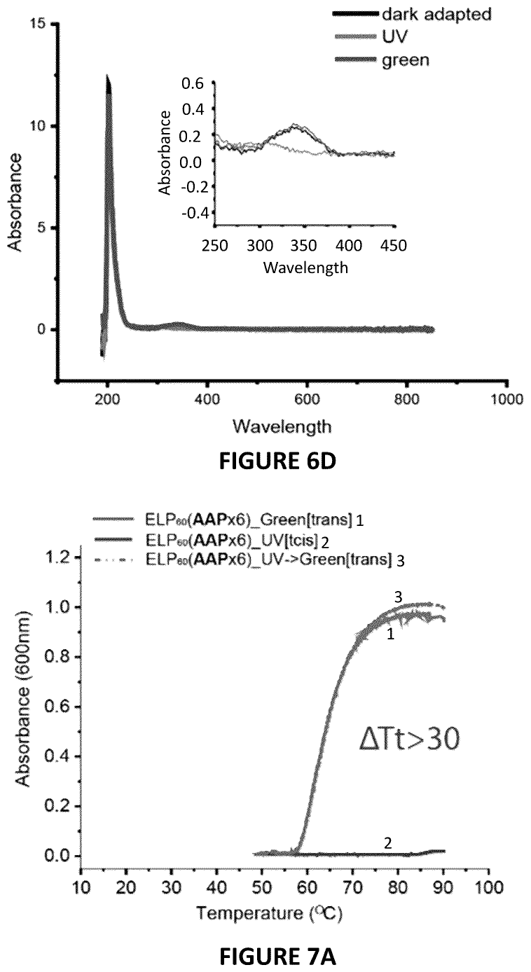

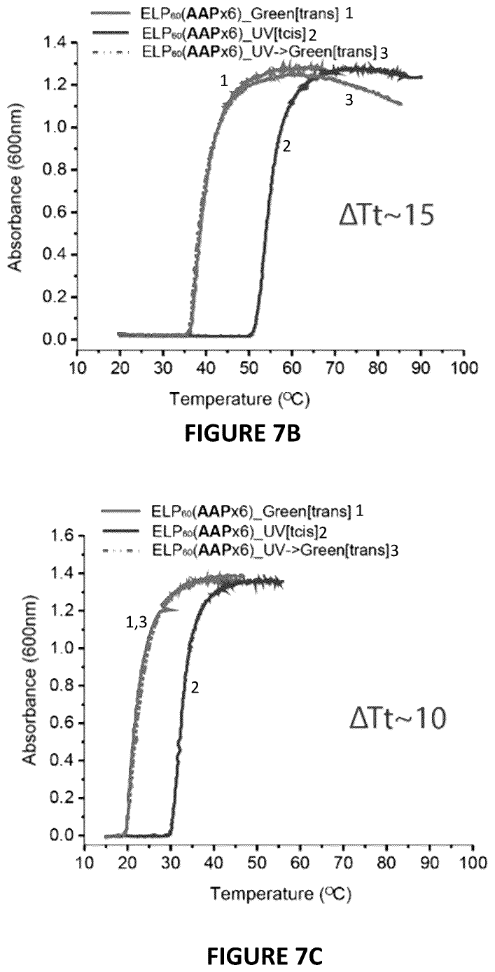

To determine the ability of a light-mediated isomerization of AAP to engender a difference in the LCST of the ELP (indicated as ΔLCST_{cis/trans}), the inventors irradiated ELP₆₀(AAP×2), ELP₆₀(AAP×6), and ELP₆₀(AAP×10) at 365 nm or 530 nm to induce isomerization to the cis (more hydrophilic) or trans (more hydrophobic) configuration, respectively. The inventors confirmed that light irradiation indeed induced the isomerization of AAP within the ELP by examining the UV-vis spectrum of ELP (AAP×2), ELP (AAP×6), and ELP (AAP×10) after irradiation with both wavelengths. Indeed, the characteristic peaks associated with the cis and trans isomers of AAP were clearly visible. Of note, the UV-vis spectra indicate near-complete cis to trans photo-isomerization as the spectra of dark-adapted and green-irradiated AAP and AAP-containing ELPs are nearly identical (FIG. 6). As expected, irradiation of AAP to produce the cis isomer generated ELPs with a higher LCST than when AAP was irradiated to produce the trans isomer, and the ΔLCST_{cis/trans} induced by the isomerization process increased with the number of incorporated instances of AAP (FIGS. 3A-3C). Of note, given the relative hydrophilicity of ELP₆₀(AAP×2), the inventors were not able to observe its LCST in water. Therefore, turbidity profiles for this protein were obtained in water supplemented with 2 M NaCl. To determine the effect of NaCl on the ΔLCST_{cis/trans}, the inventors also analyzed the light-dependent phase separation of ELP₆₀(AAP×6) in water supplemented with 1 M and 2 M NaCl. The results indicated a decrease in ΔLCST_{cis/trans} with increasing NaCl concentration (FIG. 7), that suggest that the ΔLCST_{cis/trans} of ELP₆₀(AAP×2) in water (without NaCl) may be as high as 15° C.

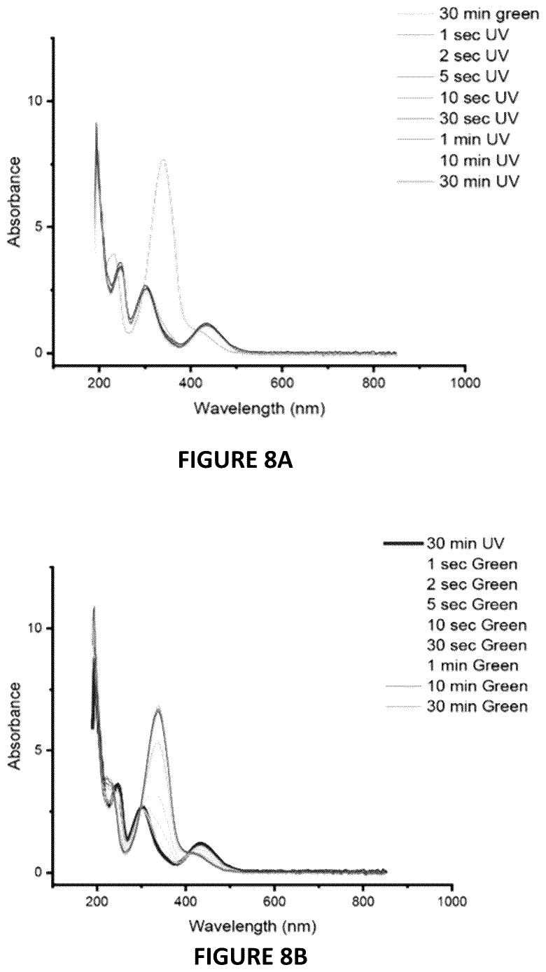

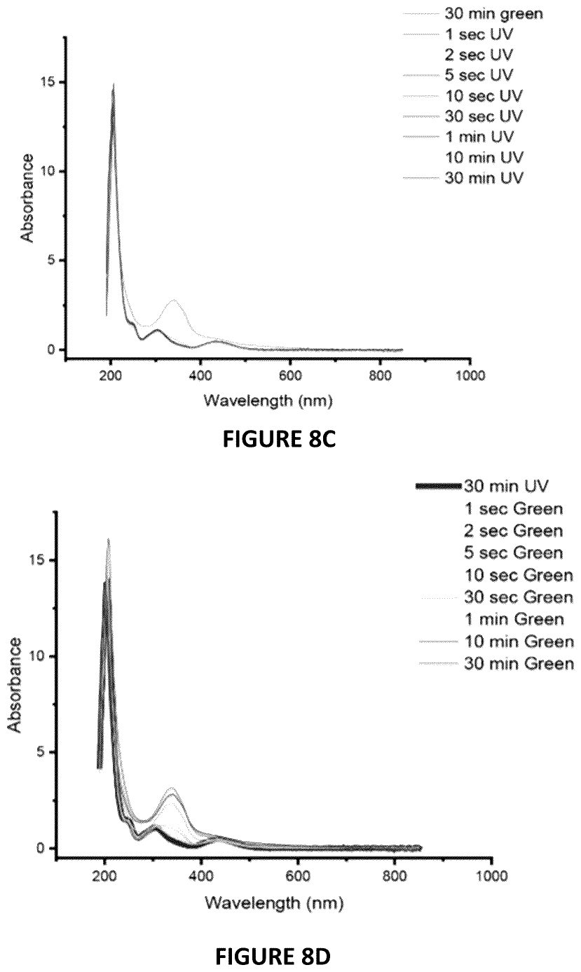

The inventors further characterized the irradiation time required for photo-switching of AAP solution and of ELP₆₀(AAP×10), which showed the largest ΔLCST_{cis/trans}. Under the current experimental conditions, as little as 60 s or 2 s of irradiation with green (370 mW) or UV (1290 mW) light, respectively, were sufficient to generate the PSS (defined as the spectrum observed after 30 min of irradiation) (FIG. 8). Accordingly, ELP₆₀(AAP×10) irradiated for only 1 min with each respective wavelength, exhibited a ΔLCST_{cis/trans} of ~31° C., which is similar to the ΔLCST_{cis/trans} of the same ELP irradiated for 30 min (FIGS. 3A and 3D). Analysis of the light-responsive behavior of ELP₆₀(Gly/AAP×10) revealed that isomerization of AAP generated an ΔLCST_{cis/trans} of ~45° C., indicating that a higher ΔLCST_{cis/trans} is expected when the overall ELP hydrophobicity is decreased (FIG. 3E). The inventors then determined the reversibility of the light-induced phase separation of ELP₆₀(AAP×10) by measuring OD₆₀₀ changes in the ELP₆₀(AAP×10) during 10 successive isothermal (38° C.) irradiation cycles (3 min or 1 min irradiation time). The resulting measurements were nearly identical throughout all 10 successive cycles (FIG. 3F), indicating that the effect of isomerization on the Tt was reproducible throughout multiple (and very short) irradiation cycles without decreasing efficiency.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A protein comprising the amino acid sequence of SEQ ID NO: 7, wherein isoleucine at position 159 (I159) is substituted with Ser or Asp.

2. The protein of claim 1, further comprising a substitution in at least one position selected from the group consisting of: 158, 162, 167, and any combination thereof, of SEQ ID NO: 7, wherein any one of: aspartate at position 158 (D158) is substituted with Cys, Met, Gly, Tyr, or Ile, leucin at position 162 (L162) is substituted with Gly, Tyr, or Asp, alanine at position 167 (A167) is substituted with Leu, Thr, or Tyr, and any combination thereof.

3. The protein of claim 1, comprising said I159 being substituted with Asp (I159D), and optionally wherein said protein comprises any one of: said D158 being substituted with Cys (D158C), said L162 being substituted with Gly (L162G), said A167 being substituted with Leu (A167L), and any combination thereof, and optionally wherein said protein comprises said D158C, said I159D, said L162G, and said A167L.

4. (canceled)

5. (canceled)

6. The protein of claim 1, further comprising a substitution in at least one position selected from the group consisting of: 32, 65, 257, 286, of SEQ ID NO: 7, wherein any one of: tyrosine at position 32 (Y32) being substituted with Gly, leucin at position (L65) being substituted with Val, arginine at position (R257) being substituted with Gly, aspartate at position 286 (D286) being substituted with Arg, and any combination thereof.

7. The protein of claim 1, comprising an amino acid sequence having not more than 98% sequence homology or identity to SEQ ID NO: 7.

8. The protein of claim 1, comprising an amino acid sequence set forth in any one of: SEQ ID Nos: 1-5, or a functional analog thereof having at least 95% homology thereto.

9. The protein of claim 1, comprising an amino acid sequence set forth in SEQ ID NO: 4.

10. The protein of claim 1, characterized by being capable of pairing a non-standard amino acid (nsAA) with a tRNA, optionally wherein said nsAA comprises a photo-switchable group, and optionally wherein said photo-switchable group is characterized by the structure: 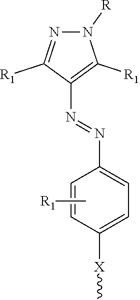
wherein R independently represents an optional substituted alkyl or H;
wherein R1 represents one or more substituents, wherein each of said one or more substituents is independently selected from the group consisting of C1-C6 alkyl, halo, NO2, CN, OH, NH2, carbonyl, CONH2, CONR′2, CNNR2, CSNR2, CONH—OH, CONH—NH2, NHCOR′, NHCSR′, NHCNR′, —NC(O)OR′, —NC(═O)NR′, —NC(═S)OR′, —NC(═S)NR′, SO2R′, SOR′, —SR′, SO2OR′, SO2N(R′)2, —NHNR′2, —NNR′, NH(C1-C6 alkyl), N(C1-C6 alkyl)2, C1-C6 alkoxy, C1-C6 haloalkoxy, hydroxy(C1-C6 alkyl), hydroxy(C1-C6 alkoxy), alkoxy(C1-C6 alkyl), alkoxy(C1-C6 alkoxy), amino(C1-C6 alkyl), CONH(C1-C6 alkyl), CON(C1-C6 alkyl)2, CO2H, CO2R′, —OCOR′, —OCOR′, —OC(═O)OR′, —OC(O)NR′, —OC(═S)OR′, —OC(═S)NR′, a heteroatom, a cyclyl, an optionally substituted cycloalkyl, optionally substituted heterocyclyl, and a combination thereof, wherein each R′ is independently selected from hydrogen, alkyl, cycloalkyl, alkenyl, aryl, heteroaryl, optionally bonded through a ring carbon, or through a heteroatom) or heterocyclyl, optionally bonded through a ring carbon, or through a heteroatom); wherein X is a bond or an alkyl; and wherein a wavy bond represents an attachment point to a backbone of the nsAA.

11. (canceled)

12. (canceled)

13. The protein of claim 10, wherein any one of: (i) said nsAA comprises arylazopyrazole (AAP); (ii) said tRNA comprises an anticodon complementary to a stop codon; and (iii) both (i) and (ii), and optionally wherein said stop codon is an amber codon (UAG).

14. (canceled)

15. (canceled)

16. (canceled)

17. An artificial or synthetic nucleic acid molecule comprising a polynucleotide encoding the protein of claim 1, and optionally wherein said artificial or synthetic nucleic acid molecule being an expression vector or a plasmid.

18. (canceled)

19. A cell comprising the protein of claim 1, and optionally wherein said cell being a bacterial cell.

20. (canceled)

21. A composition comprising the protein of claim 1, and an acceptable carrier.

22. The composition of claim 21, further comprising: (i) a tRNA comprising an anticodon complementary to a stop codon, a nsAA, or both; (ii) a tRNA comprising an anticodon complementary to a stop codon paired to a nsAA; or both (i) and (ii).

23. (canceled)

24. The composition of claim 22, wherein said stop codon is an amber codon (UAG).

25. A method for producing a protein of interest (POI) comprising a nsAA, the method comprising culturing a cell comprising a polynucleotide encoding said POI and comprising a premature stop codon, under conditions sufficient for expression of said polynucleotide, wherein said cell comprises the protein of claim 1, thereby producing the POI comprising a nsAA.

26. The method of claim 25, wherein said culturing comprises supplementing said cell with an effective amount of said nsAA.

27. The method of claim 25, wherein said producing comprises increasing production levels of said POI comprising said nsAA, compared to a control, and optionally wherein said increasing is by at least 1.5-fold compared to said control.

28. (canceled)

29. The method of claim 25, wherein said nsAA comprises a photo-switchable group.

30. The method of claim 25, wherein said photo-switchable group is characterized by the structure: 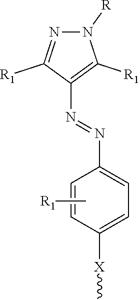
wherein R independently represents an optional substituted alkyl or H;
wherein R1 represents one or more substituents, wherein each of said one or more substituents is independently selected from the group consisting of C1-C6 alkyl, halo, NO2, CN, OH, NH2, carbonyl, CONH2, CONR′2, CNNR2, CSNR2, CONH—OH, CONH—NH2, NHCOR′,NHCSR′, NHCNR′, —NC(═O)OR′, —NC(═O)NR′, —NC(═S)OR′, —NC(═S)NR′, SO2R′, SOR′, —SR′, SO2OR′, SO2N(R′)2, —NHNR′2, —NNR′, NH(C1-C6 alkyl), N(C1-C6 alkyl)2, C1-C6 alkoxy, C1-C6 haloalkoxy, hydroxy(C1-C6 alkyl), hydroxy(C1-C6 alkoxy), alkoxy(C1-C6 alkyl), alkoxy(C1-C6 alkoxy), amino(C1-C6 alkyl), CONH(C1-C6 alkyl), CON(C1-C6 alkyl)2, CO2H, CO2R′, —OCOR′, —OCOR′, —OC(═O)OR′, —OC(═O)NR′, —OC(═S)OR′, —OC(═S)NR′, a heteroatom, a cyclyl, an optionally substituted cycloalkyl, an optionally substituted heterocyclyl, and a combination thereof, wherein each R′ is independently selected from hydrogen, alkyl, cycloalkyl, alkenyl, aryl, heteroaryl, optionally bonded through a ring carbon, or through a heteroatom) or heterocyclyl, optionally bonded through a ring carbon, or through a heteroatom); wherein X is a bond or an alkyl; and wherein a wavy bond represents an attachment point to a backbone of the nsAA.

31. The method of claim 25, further comprising a step comprising subjecting said produced POI comprising said nsAA to light at a wavelength configured to induce conformational switch of said photo-switchable group.
